(19) United States
(12) Patent Application Publication
(10) Pub. No.: US 2023/0280353 A1
(43) Pub. Date: Sep. 7, 2023

(54) DETECTION SYSTEM OF INTERACTION BETWEEN KNOWN MOLECULES AND PROTEINS BASED ON COVALENT CONNECTION AND IDENTIFICATION OR VERIFICATION METHOD THEREOF

(71) Applicant: SHANGHAI JIAO TONG UNIVERSITY, Shanghai (CN)

(72) Inventors: Shengce TAO, Shanghai (CN); Hewei JIANG, Shanghai (CN); Yunxiao ZHENG, Shanghai (CN); Hong CHEN, Shanghai (CN); Xuening WANG, Shanghai (CN)

(73) Assignee: SHANGHAI JIAO TONG UNIVERSITY, Shanghai (CN)

(21) Appl. No.: 18/024,049

(22) PCT Filed: Jan. 27, 2021
(86) PCT No.: PCT/CN2021/073899

§ 371 Date: Feb. 28, 2023

(30) Foreign Application Priority Data
Jan. 19, 2021 (CN) 202110069353.7

(51) Int. Cl. G01N 33/68; G01N 33/58

(52) U.S. Cl. CPC G01N 33/6848; G01N 33/58

(57) ABSTRACT

A detection system for the interaction between known molecules and proteins based on covalent connection and an identification or verification method thereof are disclosed. The detection system comprises: a) streptavidin-short peptide tetramer; b) PafA enzyme; and c) Biotin-modified known molecules. After a known molecule interacts with a protein, streptavidin-short peptide tetramer can efficiently capture interacting proteins of known molecules under mild conditions. Then, under the catalysis of PafA enzyme, the interaction between short peptides and known molecules is made into protein covalent binding, so that the non-covalent binding between known molecules and proteins is converted into covalent binding between streptavidin and protein, and then analysis, separation and identification are carried out. This method can capture weak interaction and instantaneous interaction on the basis of keeping the natural structure of known molecules, which can be used to verify and discover known molecules and interacting proteins.

### CROSS REFERENCE TO THE RELATED APPLICATIONS

This application is the national phase entry of International Application No. PCT/CN2021/073899 filed on Jan. 27, 2021, which is based upon and claims priority to Chinese Patent Application No. 202110069353.7 filed on Jan. 19, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBDD068-PKG_Sequence_Listing.txt, created on Feb. 10, 2023, and is 15,021 bytes in size.

### TECHNICAL FIELD

The invention belongs to the technical field of molecular biology, and relates to a detection system for the interaction between known molecules and proteins, in particular to a detection system for the interaction between known molecules and proteins based on covalent connection and an identification or verification method thereof.

### BACKGROUND

Protein is the executor of life activities. More than 80% of proteins function by interacting with other molecules, including embryonic development, cell communication, receptor-ligand binding, signal transduction and other life processes. Disordered and out-of-control protein-molecule interaction may cause cancer, neurodegenerative diseases, etc. (kesin et al., 2016. Chem. Rev., 116, 4884-4909). The interaction between small molecule or small molecule drugs and proteins in physiological processes has been widely studied in biomedical and clinical applications, which is helpful to further understand the physiological metabolic processes of the body and guide drug design and synthesis. Discovering and verifying the interaction between protein and other molecules, such as protein, DNA, RNA and small molecules, is of great significance for revealing the inherent laws of life activities at the molecular level. There are always two difficulties in the study of protein-molecule interaction. First, due to the complexity of intracellular environment, the interaction network is complicated, and each molecule has multiple interactions; Second, the interaction between protein and molecule is different in strength and duration, and weak interaction and instantaneous interaction are usually difficult to capture.

Classical protein-molecule interaction identification techniques include immunoprecipitation technology, Pull Down, ChIP technology, etc. Immunoprecipitation methods include protein-immunoprecipitation (Co-IP), Chromatin immunoprecipitation assay (ChIP) (Das P M et al., 2017. Biotechnics. 37 (6): 961-9.), RNA Immunoprecipitation(Co-IP) (Gagliardi M et al. Methods Mol Biol. 2016; 1480: 73-86.), small molecule affinity chromatography (Sleno et al. 2008, Curr Opin Chem Biol, 12, 46-54) (Sato, et al., 2010, Chem Biol, 17, 616-623), etc. ChIP and CLIP can be used to identify the interaction between DNA and RNA and protein, respectively. In living cells, formaldehyde is used to cross-link and fix DNA or RNA-protein complex. ChIP can enrich the target protein and DNA complex by antibody, while CLIP can bind specific RNA and identify the interaction protein by mass spectrometry. Co-IP enriches the target protein and its interacting proteins by antibody, which depends on the non-covalent interaction between proteins. Pull down techniques, such as GST Pull Down, RNA pull down, Small Molecule Affinity Chromatography, etc., enrich target molecules through tags linked to proteins, RNA or small molecules to obtain interacting proteins. The above two technologies are used to discover and verify protein-molecule interaction in vivo and in vitro, The method is easy to repeat, simple to operate and low in cost, but the nonspecific binding of antibody or affinity medium easily leads to high background signal, which makes it difficult to detect weak interaction and transient interaction, and the results need to be further verified by other methods (louche et al., 2017. Methods Mol. Biol., 1615, 247-255.). Protein chip (Deng et al., 2014. Cell Rep., 9, 2317-2329.) or gene chip (Hu et al., 2009, Cell, 139, 610-622) respectively make multiple protein or DNA fragments on the chip. Taking protein chip as an example, the tagged target molecules (protein, DNA, RNA, small molecules) are co-incubated with protein chip, and then the nonspecific binding is removed by washing. Subsequently, the interaction between molecules and proteins on the chip can be identified. Chip technology has the advantages of high flux, less sample consumption, short reaction time, etc. It can find the molecules interacting with the target molecules globally in one experiment, and is an efficient tool for studying molecule/protein interaction. But at the same time, this method also has some limitations. The signals detected by the chip are the results of molecular interaction in vitro. Considering the complexity and diversity of the biological environment in vivo, false positives will inevitably exist, which is also a common problem in in vitro screening methods.

Recent proximity marker systems can be used to identify interacting proteins of various molecules, such as BioID (proximity dependent biotin identification), APEX (engineered ascorbate peroxidase), PUP-IT (pupylation-based interaction tagging) (Liu et al., 2018. Nat. Methods, 15, 715-722.), CasID (Schmidtmann et al., 2016, Nucleus, 7, 476-484) and CASPEX (Myers et al., 2018, Nat Methods, 15, 437-439) can identify interacting proteins of known proteins Knowing the interaction between DNA and protein, CRIUS (Ziheng Zhang et al., 2020, Nucleic Acids Res, 1) can identify the interacting proteins of known RNA. Taking BioID as an example, the enzyme with proximity labeling function and bait protein are fused and expressed in cells, and marker molecules (such as biotin) are added into cell culture medium. Proteins adjacent/interacting with bait protein are covalently linked with marker molecules, and then capture proteins with marker molecules are identified as possible interacting proteins by mass spectrometry. CasID, CASPEX and CRIUS combine dCas9 or dCas13a proteins with existing proximal marker systems to identify proteins that interact with known DNA or RNA. Taking CRIUS as an example, PafA and dCas13a are fused and expressed, and then located to the target RNA under the action of sgRNA, and then PafA can label pup polypeptide with biotin tag on RNA binding protein; The disadvantage of this method is that it is highly dependent on the targeting efficiency of sgRNA. In the application scenario of identifying multiple RNA interactions at one time, it is necessary to design different sgRNA for different target RNA. At this time, different sgRNA targeting efficiency will easily lead to differences in the amount of PafA protein targeted and introduce systematic errors. Proximity labeling system can transform non-covalent interaction between molecules and proteins into covalent linkage between proteins and labeled molecules, and can capture weak and transient interactions in real cell environment. However, these methods need to fuse the enzyme with bait protein or dCas protein (dead Cas proteins). The large molecular weight of the enzyme may affect the original structure of bait protein, or affect the interaction between bait protein and other proteins due to steric hindrance; Moreover, the above proximal labeling system can only play a role in cells, and can not be applied to primary cells and most passages, so the above methods are not applicable to many proteins.

Existing techniques to verify the interaction between protein and DNA/RNA/small molecules include Surface Plasmon Resonance (SPR), Bio-Layer Interferometry (BLI), isothermal titration calorimetry (ITC) and so on. SPR is the gold standard for evaluating the interaction between proteins and small molecules, proteins, antibodies, etc. It can realize real-time, quantitative and highly sensitive detection without labeling proteins, and samples are easy to prepare. However, this method relies on precision instruments and is complicated to operate (Olaru et al., 2015. Crit. Rev. Anal. Chem., 45, 97-105.). BLI can be used to determine the kinetic parameters of the interaction between proteins and various molecules, which is real-time, quantitative and highly sensitive. However, this method needs to label ligand proteins or molecules in advance and depends on precision instruments. The current BLI instruments have very limited temperature control range and are not suitable for determining thermodynamic parameters (Desai et al., 2019. J. Vis. Exp. (149), e59687). Immunoprecipitation and Pull down can also be used to verify protein-protein interaction. Electrophoresis-based techniques such as Far-Western and EMSA (Cai et al., 2012, Amino Acids, 43, 1141-1146) can be used to validate protein or DNA/RNA-protein interactions, respectively. In EMSA, for example, DNA/RNA-protein complexes are formed by violet diplomatic association. Under the action of electric field, DNA/RNA-protein complexes have larger molecular weight and slower migration rate than DNA/RNA single molecules. Its disadvantages are that it depends on instruments, its operation is relatively complex and it is difficult to detect weak interactions.

In conclusion, many existing technologies need to be combined to identify and verify the interaction between proteins and molecules. The invention provides a system based on covalent connection, The core of this system is streptavidin fusion expression of four short peptides. The known molecules are modified by biotin and bound to streptavidin. The covalent connection between short peptides and capture proteins is realized through the proximity effect of PafA enzyme, which can be used to detect and verify the interaction between proteins and various molecules such as protein, DNA, RNA and small molecules.

### SUMMARY

The invention aims at overcoming the deficiencies of the prior art, and provides a detection system for the interaction between known molecules and proteins based on covalent connection and an identification or verification method thereof. The detection system can be applied to the discovery and verification of the interaction between known molecules and proteins, and realize the detection of weak interaction and instantaneous interaction on the basis of keeping the original structure and activity of known molecules, which is expected to greatly improve the sensitivity, specificity and success rate of the detection of the interaction between known molecules and proteins.

The method provided by the invention utilizes biotin to label known molecules; After the known molecules interact with other proteins, the interaction proteins of the known molecules labeled by biotin are efficiently captured under mild conditions through streptavidin coupled with Pup. Furthermore, under the catalysis of proximal labeling activity of PafA-Pup system, Pup is covalently linked with the interaction protein, so that the non-covalent binding between known molecules and the interaction protein can be converted into the covalent binding between streptavidin and the interaction protein, and the subsequent cleaning can be carried out under extremely strict conditions, thus significantly improving the specificity on the premise of ensuring sensitivity. The method of the invention can realize the capture and detection of weak interaction and instantaneous interaction on the basis of keeping the original structure and activity of known molecules.

The purpose of the invention is realized by the following technical scheme: The invention provides a detection system for the interaction between known molecules and proteins based on covalent linkage, the detection system comprising the following molecules:
a) Streptavidin-short peptide tetramer, which is fused to express four short peptides by streptavidin tetramer, wherein streptavidin can bind biotin and biotin medium, and short peptides can be covalently linked to adjacent proteins catalyzed by PafA;
b) PafA enzyme, which can covalently link specific short peptides to neighboring proteins.
c) Biotinylated known molecules capable of binding to streptavidin-short peptide tetramer.

Preferably, in the streptavidin-short peptide tetramer, the short peptide is a peptide containing 12-100 amino acids. Peptides less than 12 amino acids in length generally have no function.

Preferably, the short peptide comprises a Pup molecule or a mutant molecule thereof, and the glutamine at the end of the Pup molecule is mutated into glutamic acid, the sequence of which is as shown in SEQ ID NO: 1; The reasons are as follows: in the ubiquitin-like proteasome system of Mycobacterium tuberculosis, Dop enzyme catalyzes the deamination of glutamine at the end of Pup to form glutamic acid, and PafA catalyzes the linkage reaction between Pup (E) and substrate;

The mutation molecule of the Pup is a Pup molecule with one or more mutations, and the sequence is shown by any sequence of SEQ ID NO: 2, SEQ ID NO: 3 and SEQ ID NO: 4.

The Pup molecule is derived from any one of the genera of Mycobacterium, Corynebacterium, Streptomyces, Kocuria and Micrococcus, but is not limited thereto. For example, Pup molecules are derived from Mycobacterium tuberculosis, Mycobacterium smegmatis, Corynebacterium glutamicum, Mycobacterium leprae, Erythromycin Actinomycetes, Corynebacterium diphtheriae, Streptomyces coelicolor, Kocuria rhizophila, Micrococcus luteus, etc.

Preferably, the lysine on the surface of the streptavidin is mutated into arginine.

Preferably, the streptavidin-short peptide tetramer is a streptavidin-Pup tetramer with an amino acid sequence as shown in SEQ ID NO: 5.

Preferably, seven lysine mutations on the surface of the PafA enzyme are arginine, and the mutation sites are K162R, K202R, K320R, K361R, K423R, K435R and K446R. The surface of the mutated PafA enzyme does not contain lysine, thus avoiding non-specific covalent connection of Pup molecules. The mutated PafA enzyme sequence is shown in SEQ ID NO: 7.

The PafA enzyme is derived from, but is not limited to, any one of Mycobacterium, Corynebacterium, Streptomyces, Kocuria and Micrococcus. For example, PafA enzyme comes from Mycobacterium tuberculosis, Mycobacterium smegmatis, Corynebacterium glutamicum, Mycobacterium leprae, Erythromycin Actinomycetes, Corynebacterium diphtheriae, Streptomyces coelicolor, Kocuria rhizophila, Micrococcus luteus, etc.

Preferably, the known molecules of the biotin modification include any one or more of proteins, DNA, RNA, small molecules.

Preferably, the protein comprises at least one of a protein, a peptide, a modified peptide, an antibody, a lectin, and can bind to a streptavidin-short peptide;

The RNA comprises at least one of messenger RNA, ribosome RNA, long chain non-coding RNA and non-coding small RNA, which can be combined with streptavidin-short peptide;

The DNA comprises at least one of double-stranded DNA and closed circular DNA, which can be combined with streptavidin-short peptide;

The small molecule comprises at least one of bioactive oligonucleotides, amino acids, vitamins, secondary metabolites of animal and plant microorganisms and chemically synthesized small molecules, which can be combined with streptavidin-short peptides.

More preferably, the size of the small molecule is 50-1500 Da.

Preferably, the biotin modification sites of the protein, DNA and RNA are N-terminal, C-terminal or any other sites, and the biotin decoration sites of the small molecules are optional non-critical active sites.

The invention also provides a method for identifying interactions between known molecules and proteins according to the aforementioned detection system, comprising the following steps:
A. The known biotinylated molecules and the sample to be tested are fully mixed and incubated at 25-35° C. for 0-1h;
B. Streptavidin-short peptide tetramer is added to the mixture treated in step A, thoroughly mixed and incubated at 25° C. to 35° C. for 0-1 h;
C. PafA enzyme is added into the mixture after step B treatment, thoroughly mixed and incubated at 25° C.-35° C. for 1 min-6 h;
D. The biotin-labeled affinity medium is added to the mixture after step C treatment to isolate streptavidin-short peptide and its associated proteins;
E. Mass spectrometry identification.

Preferably, the sample to be tested comprises at least one of a protein, a living cell or tissue, a membrane protein, a cell lysate, and a tissue lysate.

Preferably, the biotin labeled affinity medium includes, but is not limited to, biotin magnetic beads, biotin agarose beads.

The method is used for detecting the interaction between known molecules and proteins in the sample to be tested.

The invention also provides a method for verifying the interaction between a known molecule and a protein according to the aforementioned detection system, comprising the following steps:
S1. The known molecules to be verified are thoroughly mixed with the proteins to be verified, and incubated at 25° C. to 35° C. for 0-1h;
S2. Streptavidin-short peptide tetramer is added to the mixture after step S1 treatment, thoroughly mixed and incubated at 25° C. to 35° C. for 0-1h;
S3. PafA enzyme is added into the mixture treated in step S2, thoroughly mixed and incubated at 25° C.-35° C. for 1 min-6h.
S4. Western blot analysis is used to detect the interaction between known molecules to be verified and proteins to be verified.

Preferably, the known molecule to be verified is a known molecule modified by biotin, including any one or more of protein, DNA, RNA, small molecule;

The protein comprises at least one of a protein, a peptide, a modified peptide, an antibody and a lectin;

The RNA comprises at least one of messenger RNA, ribosome RNA, long chain non-coding RNA and non-coding small RNA;

The DNA comprises at least one of double-stranded DNA and closed circular DNA; The small molecule comprises at least one of bioactive oligonucleotides, amino acids, vitamins, secondary metabolites of animal and plant microorganisms and chemically synthesized small molecules in organisms.

Compared with the prior art, the invention has the following beneficial effects:
1. The system for detecting the interaction between known molecules and proteins based on covalent connection provided by the invention utilizes the strong affinity between biotin and streptavidin to realize the adjacent covalent connection between known molecules and Pup molecules. When the known molecules are protein, DNA and RNA, compared with the prior proximity labeling technology, i.e. the method of using fusion expression of the modified enzyme bait protein or dCas protein, the invention avoids the steric hindrance problem between the modified enzyme and the bait protein or dCas protein to a certain extent, can maintain the structure and activity of the modified enzyme and the bait protein, and can detect protein interaction more accurately.
2. The known molecule-protein interaction detection system of the invention converts the non-covalent interaction between the known molecule and the protein into the covalent interaction between the Pup molecule and the capture protein through lysine, which can realize the capture of weak interaction and instantaneous interaction, and has high sensitivity and low false positive.
3. The system for detecting the interaction between known molecules and proteins provided by the invention is applied to the verification of the interaction between known molecules and proteins, Western blot analysis can be used to read the results through the obvious migration on the gel caused by the covalent coupling of the interacting protein with streptavidin-short peptide tetramer, which is simple to operate and does not depend on expensive instruments.

### BRIEF DESCRIPTION OF THE DRAWINGS

Other features, objects, and advantages of the present invention will become more apparent by reading the detailed description of non-limiting embodiments with reference to the following drawings:

FIG. 1 is a schematic diagram of the application of the present invention in the discovery of interacting proteins; SA is a streptavidin tetramer, Pup is a short peptide, PafA_{7KR} is a mutant PafA enzyme, Bait is a protein to be studied, Prey is a captured interacting protein, and Biotin agarose is a biotinylated agarose bead.

FIG. 2 is a schematic diagram of the application of the present invention in the verification of interacting proteins; Among them, A and B are interacting proteins to be verified, and A protein is biotin modified protein.

FIGS. 3A-3B show that GFP-Pup (E) is self-connected under the action of PafA enzyme activity.

FIG. 4A is the schematic diagram of mutation site and amino acid sequence of SAₘ-Pup^{E}; FIG. 4B is the detection of biotin binding activity of SAₘ-Pup^{E}; and FIG. 4C is the detection of stability of SAₘ-Pup^{E} binding biotin agarose beads in high salt buffer.

FIG. 5A is a schematic diagram of PafA_{7KR} enzyme mutation site and amino acid sequence; FIG. 5B is self-Pup binding detection of PafA7_{KR} enzyme of itself; and FIG. 5C is the detection ability of PafA7_{KR} enzyme for substrate modified with Pup.

FIG. 6A is the result of verifying the interaction between CheZ and CheAs (wild type) at different concentrations; FIG. 6B is the result of verifying the interaction between CheZ and different mutant CheAs (WT, L126A, L123A); and FIG. 6C is the covalent linkage site between CheAs (wild type) and SAₘ-Pup^{E} detected by mass spectrometry.

FIG. 7A is a schematic diagram for detecting CobB-interacting proteins; FIG. 7B is a flowchart for detecting CobB-interacting proteins; and FIG. 7C shows the comparison between CobB interacting protein obtained by this method and the existing results.

FIG. 8A is the purification result of CobB and some interacting proteins; FIGS. 8B-8F show the interaction between protein captured by BLI detection and CobB; and FIGS. 8G-8H are the verification of deacetylation function of CobB interacting with VacB and DksA.

FIG. 9A is a schematic diagram of cell surface receptor for detecting PD-1 protein; FIG. 9B is a flowchart of cell surface receptor for detecting PD-1 protein; and FIG. 9C is a diagram for verifying the interaction between PD-1 protein and its cell surface receptor PD-L1.

FIG. 10A is a flow chart for detecting the interacting protein of SARS-CoV-2 protein; FIG. 10B is a comparison between the interacting protein of SARS-CoV-2 protein obtained by this method and the existing results; and FIG. 10C is a demonstration of the interaction between SARS-CoV-2 protein ORF9b and human protein TOM70.

FIG. 11 is a schematic diagram of the application of the present invention in the discovery of RNA-protein interaction; SA is streptavidin tetramer, Pup is short peptide, PafA7_{KR} is mutant PafA enzyme, RNA is biotinylated RNA, Prey is captured interacting protein, Biotin agarose is biotinylated agarose bead.

FIG. 12 is a schematic diagram of the application of the present invention in the verification of RNA-protein interaction; Among them, RNA is biotinylated RNA and Prey is captured interacting protein.

FIGS. 13A-13C are the results of verifying the interaction between m6A and YTDHF1, YTDHF2 and YTDHF3.

FIG. 14 is a schematic diagram of the application of the present invention in the discovery of DNA-protein interaction; SA is streptavidin tetramer, Pup is short peptide, PafA7_{KR} is mutant PafA enzyme, DNA is biotinylated DNA, Prey is captured interacting protein, Biotin agarose is biotinylated agarose bead.

FIG. 15 is a schematic diagram of the application of the present invention in the verification of DNA-protein interaction; DNA is biotinylated DNA and Prey is the captured interaction protein.

FIG. 16 is the result of verifying the interaction between different DNA systems and EthR.

FIG. 17 is the result of verifying the interaction between different DNA fragments and RutR.

FIG. 18 is the result of verifying the interaction between different DNA systems and GCN4.

FIG. 19 is an application schematic diagram of the present invention in the discovery of interaction between small molecules and proteins; SA is streptavidin tetramer, Pup is a short peptide, PafA7_{KR} is a mutant PafA enzyme, SM is a small molecule to be studied, Prey is a captured interacting protein, and Biotin agarose is a biotinylated agarose bead.

FIG. 20 is a schematic diagram of the application of the present invention in the verification of interacting small molecules and proteins; Where A is a small molecule modified by biotin and B is a protein to be verified.

FIGS. 21A-21C show the interaction between different small molecules and proteins, in which FIG. 21A is the result of verifying the interaction between small molecule C-di-GMP and ETHR with different concentrations; FIG. 21B is the result of verifying the interaction between small molecule C-di-GMP and CSP series short peptides; and FIG. 21C is the result of verifying the interaction between small molecule Rapamycin and FKBP12.

Wherein, “SPIDER” shown in each drawing represents the abbreviation of the detection system of the present invention.

### DETAILED DESCRIPTION OF THE EMBODIMENTS

The present invention is described in detail below in connection with specific embodiments. The following embodiments will facilitate a further understanding of the invention by those skilled in the art but do not limit the invention in any form. It should be noted that several modifications and modifications may be made to those of ordinary skill in the art without departing from the concept of the invention. All these belong to the protection scope of the present invention.

The principle of the invention is described as follows:

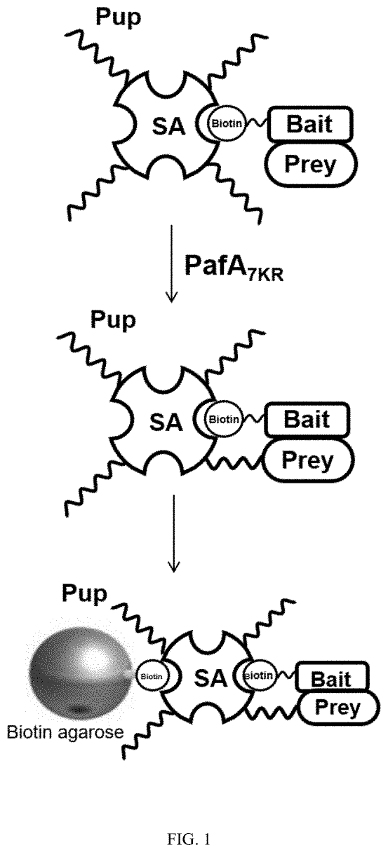

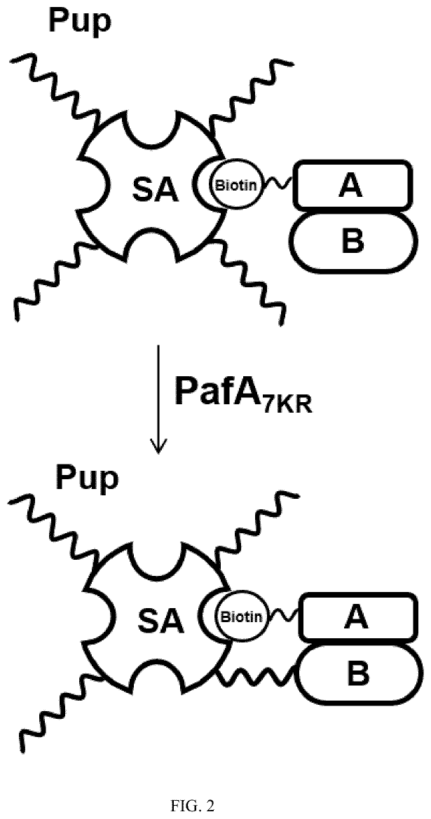

1. The schematic diagram of protein interaction is shown in FIG. 1. Biotin-labeled bait protein binds to streptavidin-Pup tetramer (SA-Pup). When the protein in the system interacts with bait protein, free PafA7_{KR} enzyme covalently links the C-terminal of Pup to the interacting protein. Biotin-labeled affinity medium was used to enrich the capture protein of SA-Pup covalently linked, that is, the interaction protein of bait protein.

2. The schematic diagram for verifying protein interaction is shown in FIG. 2. Biotinylated protein A binds to SA-Pup. When protein B interacts with protein A, PafA7_{KR} enzyme free in the system covalently links the C terminal of Pup to protein B.

3. The schematic diagram of the interaction between RNA and protein is shown in FIG. 11. Biotin-labeled RNA binds to streptavidin-Pup tetramer (SA-Pup). When the protein in the system interacts with RNA, the free PafA7_{KR} enzyme covalently links the C-terminal of Pup to the interacting protein. Biotin-labeled affinity media were used to enrich the capture protein of SA-Pup covalently linked, that is, the interaction protein of RNA.

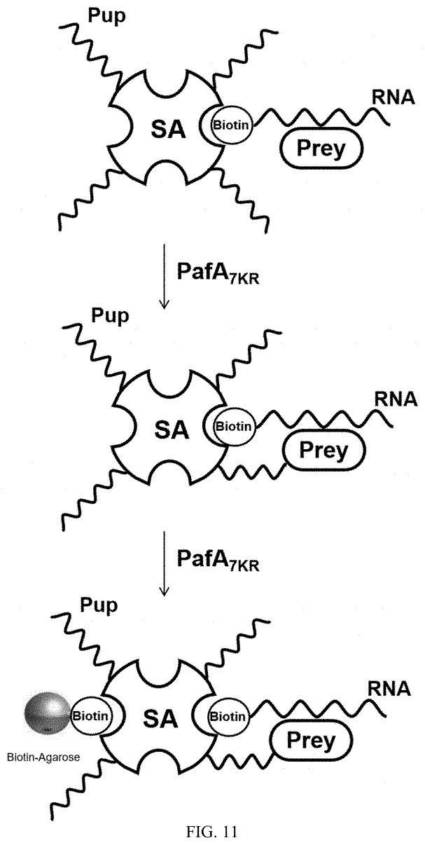

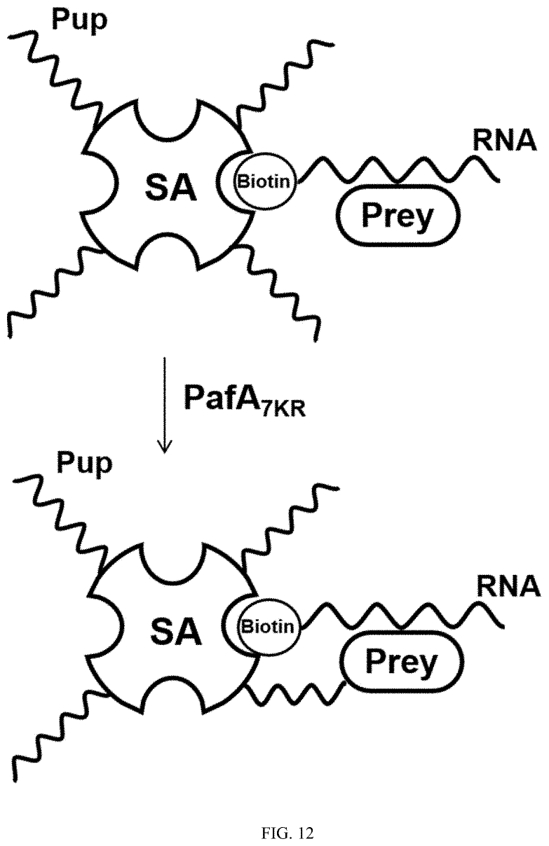

4. The schematic diagram for verifying the interaction between RNA and protein is shown in FIG. 12. Biotinylated RNA binds to SA-Pup. When the protein interacts with RNA, PafA7_{KR} enzyme in the system covalently connects the C terminal of Pup to the protein.

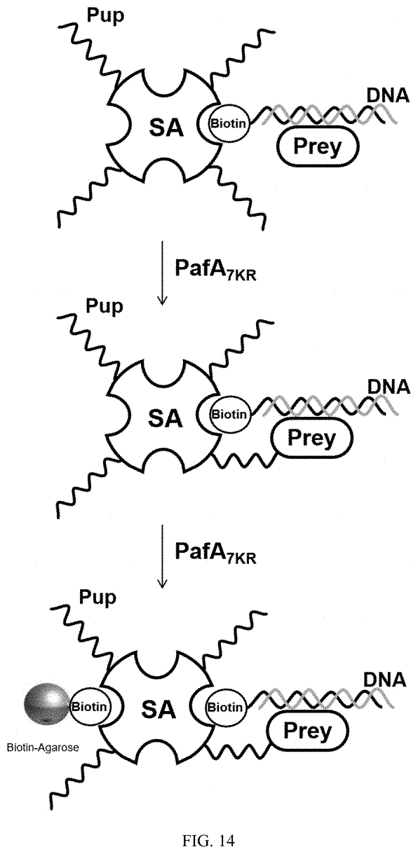

5. The schematic diagram of the interaction between DNA and protein is shown in FIG. 14. Biotin-labeled DNA binds to streptavidin-Pup tetramer (SA-Pup). When the protein in the system interacts with DNA, the free PafA7_{KR} enzyme covalently links the C-terminal of Pup to the interacting protein. Biotin agarose beads were used to enrich the capture protein of SA-Pup covalently linked, that is, the interaction protein of DNA.

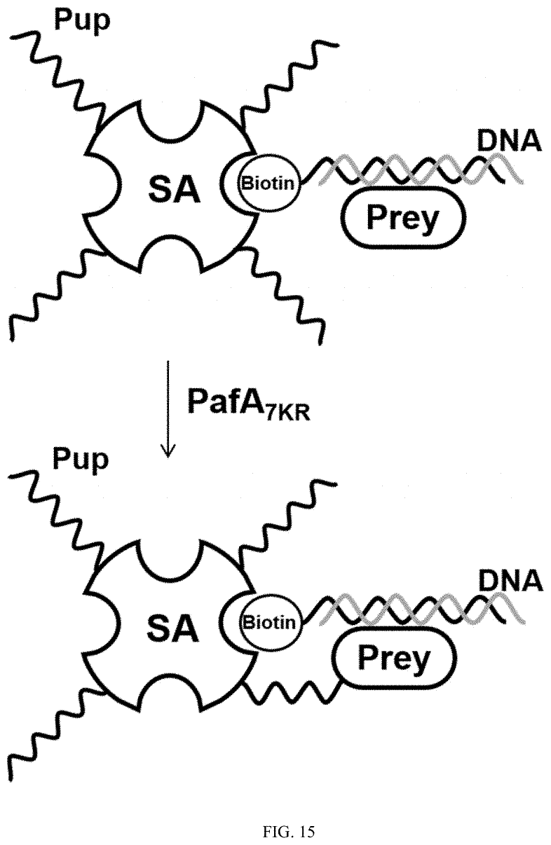

6. The schematic diagram for verifying the interaction between DNA and protein is shown in FIG. 15. Biotinylated DNA binds to SA-Pup. When the protein interacts with DNA, PafA7_{KR} enzyme in the system covalently connects the C terminal of Pup to the protein.

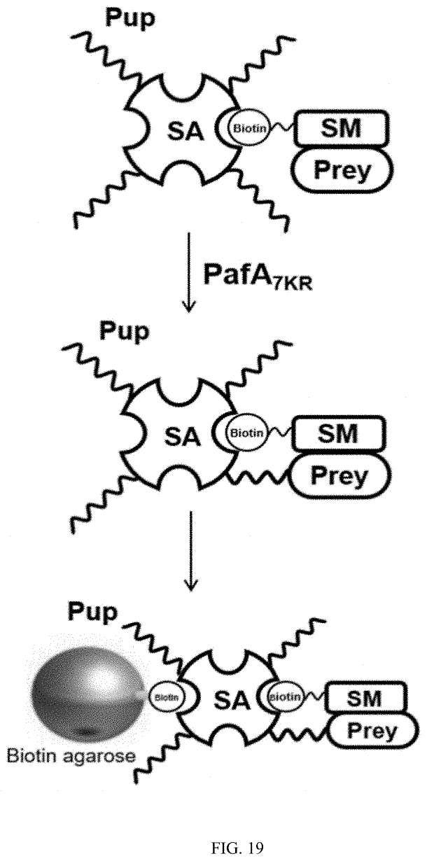

7. The schematic diagram of the interaction between small molecules and proteins is shown in FIG. 19. The biotin-labeled bait molecule binds to streptavidin-Pup tetramer (SA-Pup). When the protein in the system interacts with the bait molecule, the free PafA7_{KR} enzyme covalently links the C-terminal of Pup to the interacting protein. Biotin-labeled affinity medium was used to enrich the capture protein of SA-Pup covalently linked, that is, the interaction protein of bait small molecules.

8. The schematic diagram for verifying the interaction between small molecules and proteins is shown in FIG. 20. The biotinylated bait molecule binds to SA-Pup. When the protein interacts with the bait molecule, the free PafA7_{KR} enzyme in the system covalently connects the C terminal of Pup to the protein.

In the following embodiments, the adopted E. coli BL31 (DE3) strain was purchased from TransGen Biotech Co., Ltd., HEK293T cells were purchased from the Cell Bank of Chinese Academy of Sciences. The vector pET28a, pTrc99a and pET32a are commonly used plasmids in laboratory. Biotin agarose beads were purchased from Sigma-Aldrich Company. Nickel column was purchased from Zhongke Senhui Microsphere Technology (Suzhou) Co., Ltd., Annealing Buffer (5×) was purchased from Biyuntian Biotechnology Co., Ltd., biotinylated C-di-GMP was purchased from biolog Company, biotinylated lenalidomide small molecule was presented by Dong Jiajia, Shanghai Institute of Organic Chemistry, Chinese Academy of Sciences, and biotinylated Rapamycin was presented by Dang Yongjun, School of Basic Medicine, Fudan University.

### Embodiment 1: Reactivity Verification of PafA and Pup (E)

1. Obtain GFP-Pup (E) and Pup (E)-GFP Proteins

Pup (E) refers to the mutation of glutamine (Q) at the C terminal of wild-type Pup molecule (sequence as shown in SEQ NO. 1) to

Glutamic acid (E). Pup (E) was fused and expressed at the N-terminal and C-terminal of GFP protein, respectively. GFP-Pup (E) and Pup (E)-GFP were constructed on pET28a and transformed into E. coli BL21 (DE3) strain, respectively, without a 6×His tag attached to the terminal of Pup (E). GFP-Pup (E) and Pup (E)-GFP proteins were purified by nickel column after IPTG was added in 1 L of bacteria medium when OD₆₀₀ was about 0.6 and induced overnight at 18° C.

2. Obtain PafA Enzyme

The wild-type PafA sequence is shown in SEQ NO. 6. PafA was linked to the pTrc99a vector and transformed into E. coli BL21 (DE3) strain, in which the C-terminal of PafA was linked to a 6×His tag. PafA enzyme was obtained by adding IPTG at 18° C. overnight when OD600 was about 0.6 and purified by nickel column.

3. Verify the Reactivity of PafA and Pup (E)

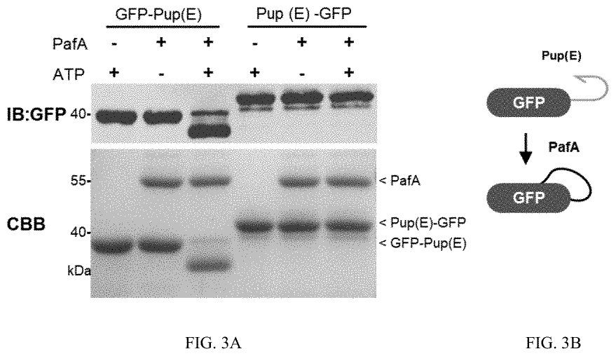

10 μL enzyme activity reaction system was prepared. The protein concentration ratio was as follows: GFP-Pup (E) or Pup (E)-GFP (10 μM), PafA (0.5 μM), ATP (5 mM). The insufficient volume was filled with reaction buffer (50 mM Tris, PH 7.5, 100 mM NaCl, 20 mM MgCl₂, 10% (v/v) glycerol), and the reaction was carried out at 30° C. for 6h. SDS-PAGE and Coomassie Brilliant Blue staining were used. As shown in FIGS. 3A-3B, the GFP-Pup (E) band migrates downward, indicating that GFP-Pup (E) is self-connected, PafA and Pup (E) have Pupification reactivity, and Pup (E)-GFP band does not migrate, indicating that PafA can only covalently connect Pup (E) molecule to the substrate through the C-terminal of Pup (E) molecule.

### Embodiment 2 Modification and Activity Verification of Streptavidin-Pup

1. Obtain the Modified Streptavidin-Pup Tetramer Protein

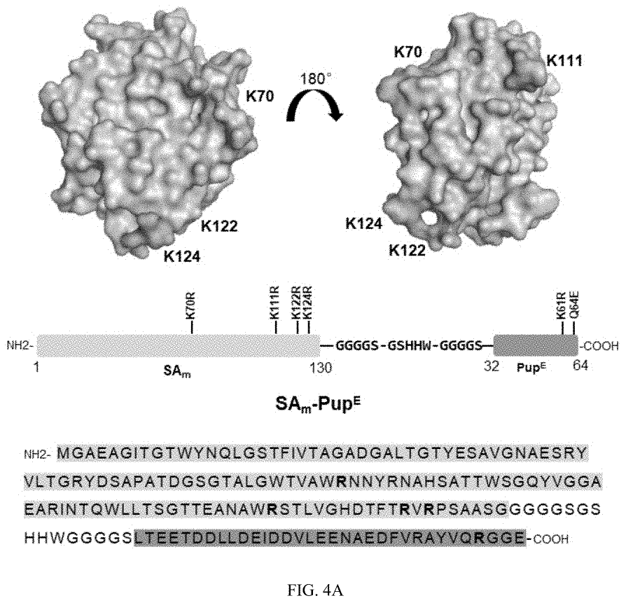

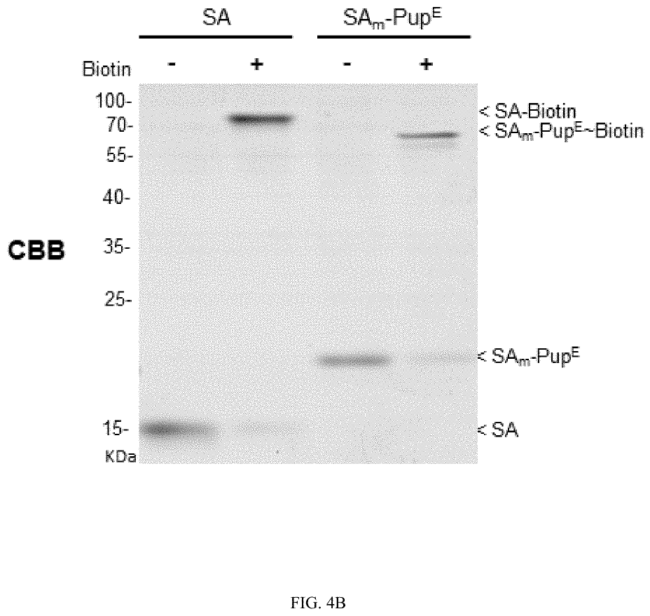

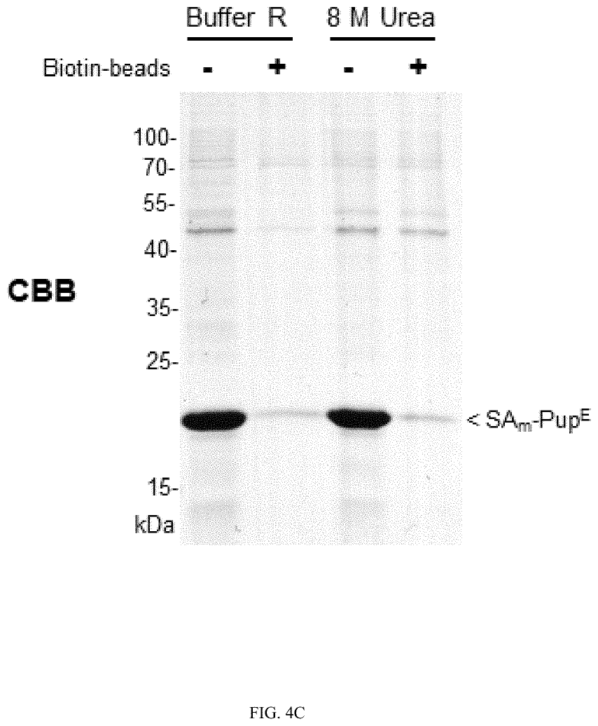

To avoid streptavidin-Pup self-linking (Pup sequence is shown in SEQ ID NO: 1), the surface of streptavidin protein and lysine of Pup molecule are mutated into arginine, and the mutated streptavidin-Pup tetramer (SAₘ-Pup^{E}) amino acid sequence (SEQ ID NO: 5) is shown in FIG. 4A (where Pup^{E} sequence is shown in SEQ ID NO: 2). SAₘ-Pup^{E} was constructed into pET28a vector and transformed into E. coli BL31 (DE3) strain. The SAₘ-Pup^{E} protein was purified by the inclusion body renaturation method (Michael T. Jacobsen et al., 2017. Cell. Chem. Bio., 2017 Aug. 17; 24 (8): 1040-1047) when OD₆₀₀≈0.6, IPTG with a final concentration of 0.5 mM was added 1 L bacteria solution cultured, and induced at 37° C. for 4 h. The SAₘ-Pup^{E} protein was purified by the inclusion body renaturation method (Michael T. Jacobsen et al., 2017. Cell. Chem. Bio., 2017 Aug. 17; 24 (8): 1040-1047).

2. Biotin Binding Activity of SAₘ-Pup^{E} was Detected as Shown in FIG. 4B.

The SAₘ-Pup^{E} protein and wild-type streptavidin (SA) purified in step 1 were mixed with biotin (A600078) and incubated at room temperature for 1 hour, and then detected by SDS-PAGE. As can be seen in FIG. 4B, SAₘ-Pup^{E} exhibits the same biotin-binding activity as wild-type streptavidin.

3. The Stability of the Binding of SAₘ-Pup^{E} to Biotin Agarose Beads was Examined, as Shown in FIG. 4C.

The SA-Pup purified from Step 1 was added into Buffer R (50 mM Tris, PH 7.5, 100 mM NaCl, 20 mM MgCl₂, 10% (v/v) glycerol) and high salt buffers (50 mM Tris-HCl, PH 8.0, 8 M urea, 15 mM DTT, 1 mM EDTA, PH 8.0) containing 8 M urea, respectively. After mixing, the supernatant was absorbed, and then biotin agarose beads were added to rotate and incubate at room temperature for 1 hour, and then the supernatant was absorbed. The supernatant was detected by SDS-PAGE. As shown in FIG. 4C, SAₘ-Pup^{E} and biotin agarose beads can still bind stably in high salt buffer.

The embodiment also provides a modified streptavidin-Pup tetramer protein, which is prepared by the method of step 1, with the only difference that the corresponding streptavidin-Pup tetramer proteins SAₘ-Pup^{E-1} and SAₘ-Pup^{E-2} are prepared by adopting the mutant molecular sequence of Pup as shown in SEQ ID NO: 3 or SEQ ID NO: 4.

### Embodiment 3 Modification and Activity Verification of PafA Enzyme

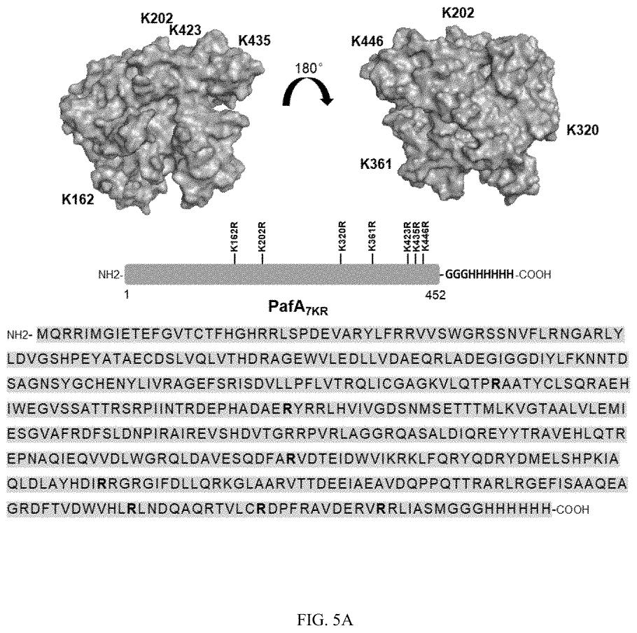

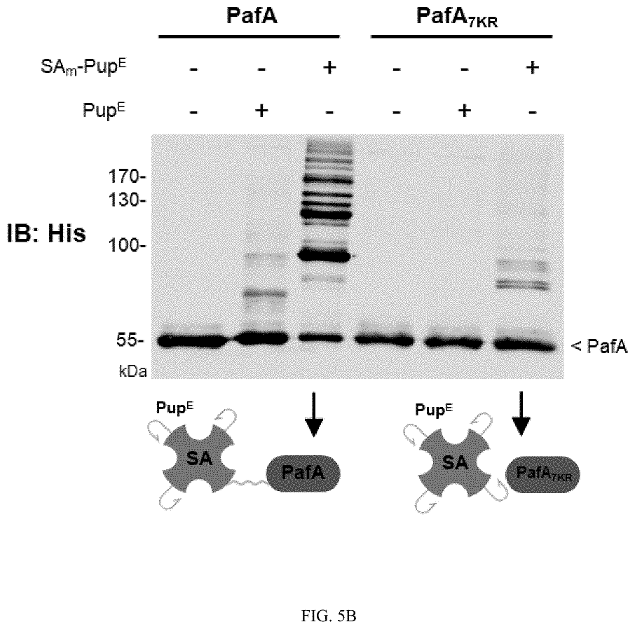

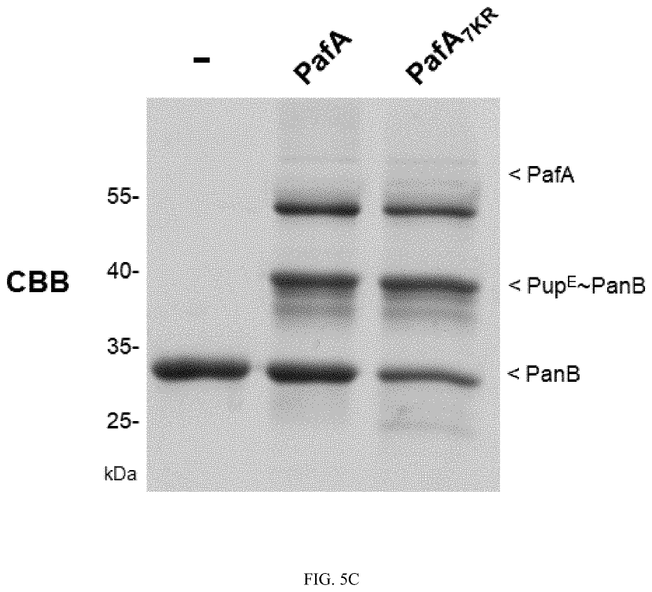

1. Obtain the Modified PafA Enzyme, and the Sequence (SEQ ID NO: 7) Shown in FIG. 5A.

In order to avoid self-linking of PafA (sequence as shown in SEQ ID NO: 6), seven lysine sites on its surface were mutated into arginine, and the mutation sites were K162R, K202R, K320R, K361R, K423R, K435R and K446R. PafA (named PafA7_{KR}) with seven point mutations constructed by QuikChange 0 site-directed mutagenesis kit (Agilent Company) was connected to pTrc99a vector and transformed into E. coli BL21 (DE3) strain, in which PafA7_{KR} C terminal was connected with a 6×His tag. PafA7_{KR} enzyme was obtained by adding IPTG, inducing overnight at 18° C. when OD600 was about 0.6, and purifying by nickel column.

2. Detect the Pupification Activity of PafA7_{KR} Enzyme on its Pup Modification, as Shown in FIG. 5B.

PafA7_{KR} enzyme was incubated with SAₘ-Pup^{E} or Pup^{E} at 30° C. for 4 h, and its Pupping degree was detected by WB. As shown in FIG. 5B, compared with wild-type PafA, PafA7_{KR} significantly reduced its Pupping connection, and only a small amount of self-connection occurred.

3. Detect the Ability of PafA7_{KR} to the Pup Modification of Substrate, as Shown in FIG. 5C.

PafA7_{KR} was incubated with Pup and PanB at 30° C. for 6h and detected by SDS-PAGE. As shown in FIG. 5C, PafA7_{KR} exhibited the same substrate Pupping efficiency as wild-type PafA.

### Embodiment 4 Verification of the Interaction Between CheAs Protein and CheZ Protein

1. Obtain Biotin Modified Protein CheZ.

The CheZ sequence with Avi tag was constructed onto pET32a vector, and BirA enzyme with biotin labeling function was constructed onto pET28a vector, and the two plasmids were co-transformed into E. coli BL21 (DE3) strain. The biotin-modified protein CheZ was obtained by incubating 1 L bacteria solution, OD600≈0.6, adding IPTG, inducing overnight at 18° C., and purifying by nickel column.

2. Obtain the Protein CheAs to be Verified

CheAs wild type and mutant (L126A, L123A) sequences were linked with 6×His and Flag tags to obtain CheAs-Flag-His sequences, in which 6×His tags were used for protein purification and Flag tags were used for Western blot detection. The CheAs-Flag-His sequence was constructed on pET28a vector and transformed into E. coli BL21 (DE3) strain. When OD600 was 0.6, IPTG was added, induced at 37° C. for 3h, and CheAs protein was purified by nickel column.

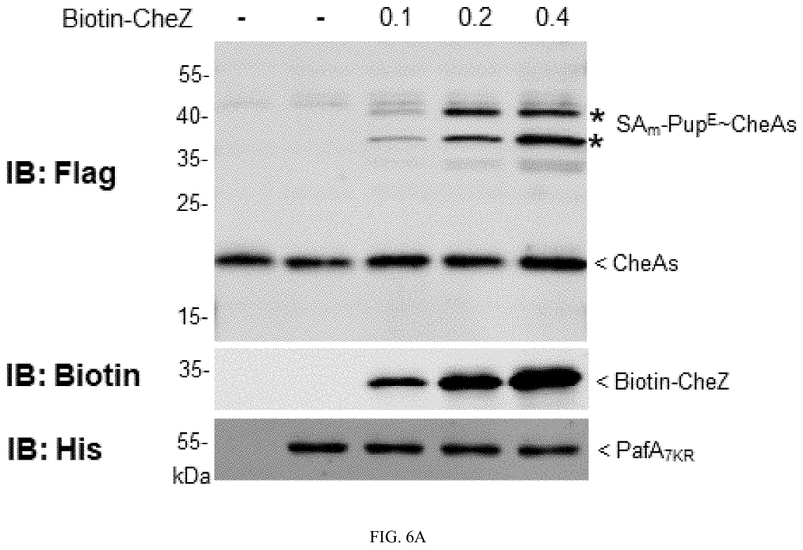

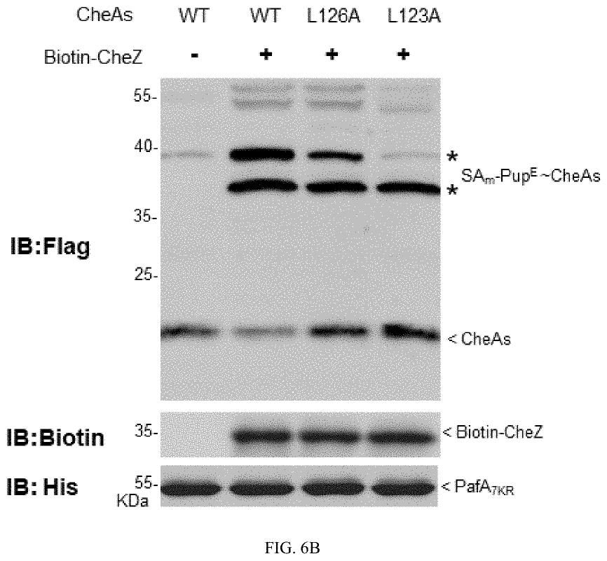

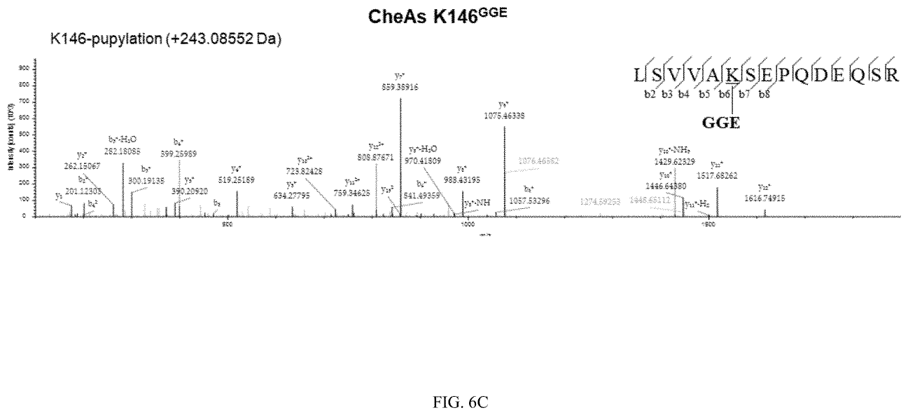

3. Verify the Interaction Between CheZ Protein at Different Concentrations and Wild-Type CheAs Protein, as Shown in FIG. 6A.

Biotinylated protein CheZ was mixed with wild-type protein CheAs (0.2 μM) and SAₘ-Pup^{E}. The concentration gradients of biotinylated protein CheZ are set to 0, 0.1 μM, 0.2 μM and 0.4 μM. PafA7_{KR} (10 mM) and ATP (5 mM) were added into the system, and then incubated at 30° C. for 6h. Flag antibody was used for Western blot analysis. If there is no interaction between protein CheAs and protein CheZ, CheAs bands (truncated type, about 19 kDa) were detected; If there is interaction between the two proteins, the complex bands of CheAs and SAₘ-Pup^{E} monomer were detected. As shown in FIG. 6A, as CheZ concentration increases, the detected complex bands become thicker, indicating that the present invention verifies that protein interactions are in a concentration-dependent manner.

4. Verify the Interaction Between CheZ Protein and Different Mutant CheAs Protein, as Shown in FIG. 6B.

The biotinylated protein CheZ (0.4 μM) was well mixed with the protein CheAs (0.2 μM) and SAₘ-Pup^{E}. CheAs include wild type (WT) and mutant type (L126A, L123A). PafA7_{KR} (10 mM) and ATP (5 mM) were added into the system, mixed well and incubated at 30° C. for 6h. Flag antibody was used for Western blot analysis. As shown in FIG. 6B, the affinity of CheAs to CheZ decreased after mutation, and the complex bands detected were finer, indicating that the present invention can be used to verify protein interactions with different affinities.

5. Identify the CheAs Protein-Formed Complex by Mass Spectrometry, as Shown in FIG. 6C.

LC-MS/MS detection of the CheAs protein-formed complex showed that the C-terminal of SAₘ-Pup^{E} was linked to multiple CheAs sites, including K146 site, as shown in FIG. 6C.

This example also verified that the streptavidin-Pup tetramer protein prepared using the Pup (E) of example 1 (the preparation method is the same as that of example 2, except that Pup^{E} is replaced by Pup (E)) was used to verify the interaction between CheAs protein and CheZ protein, and the results were similar to those of FIGS. 6A-6C.

This example also verified that SAₘ-Pup^{E-1} and SAₘ-Pup^{E-2} as described in Example 2 were used to verify the interaction between CheAs protein and CheZ protein, and the results were similar to those of FIGS. 6A-6C.

### Embodiment 5 Detection of Interacting Proteins of CobB

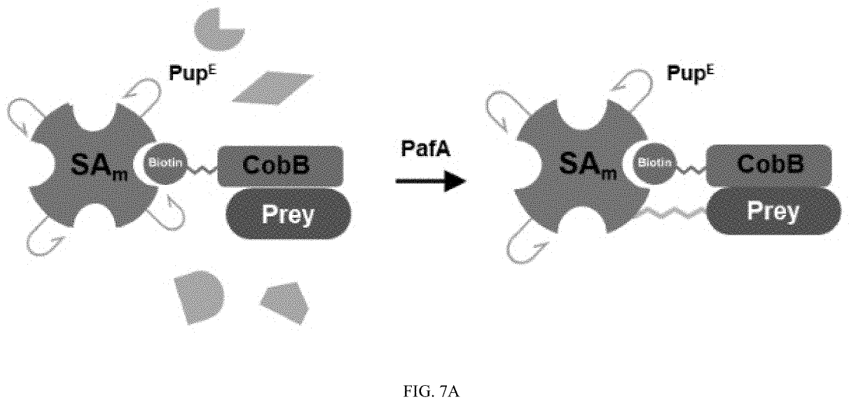

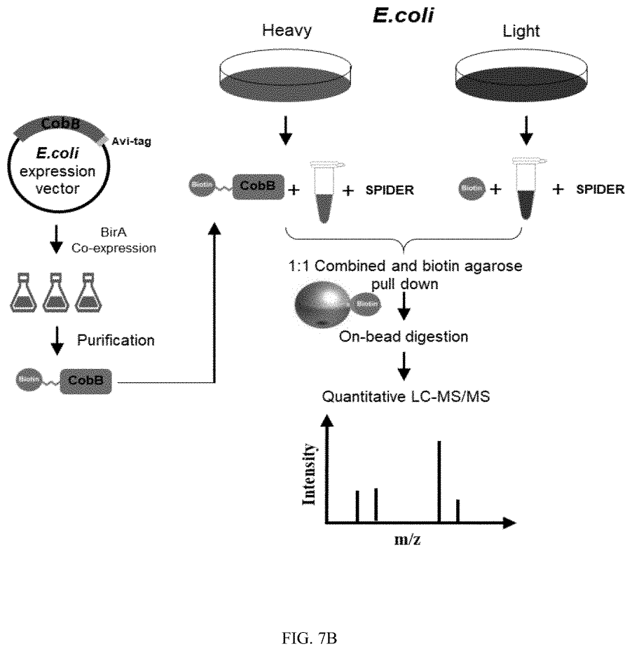

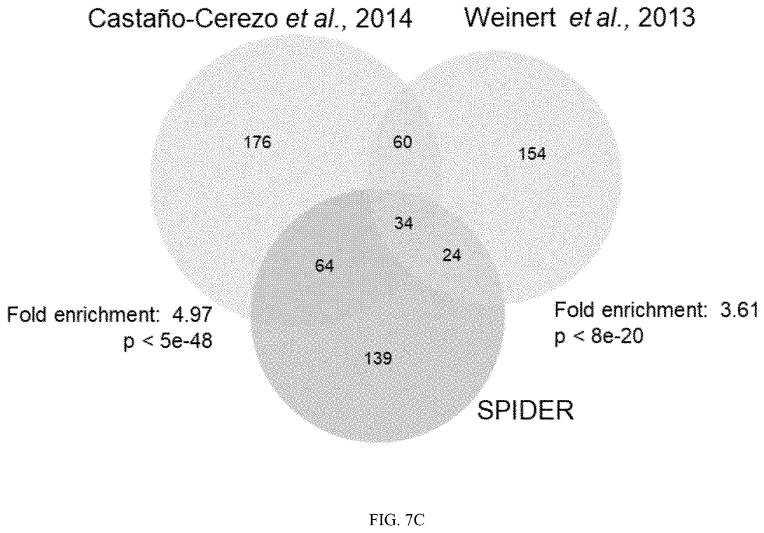

1. The principle of using this method to detect CobB interacting proteins is shown in FIG. 7A.

When biotinylated CobB protein binds to SAₘ-Pup^{E}, PafA7_{KR} exerts proximity labeling activity to covalently link the C-terminal of SAₘ-Pup^{E} to the interacting protein of CobB when the protein in cell lysate interacts with CobB.

2. The Flow of Detecting CobB Interacting Proteins Using this Method is Shown in FIG. 7B.

Firstly, the biotinylated CobB protein was constructed and reacted with SAₘ-Pup^{E}, PafA7_{KR} enzyme and E. coli SLIAC (Lys/Arg relabeled) cell lysate (experimental group). In the control group, SLIAC relabeled cell lysate was replaced by E. coli common cell lysate. SAₘ-Pup^{E} and its covalently linked capture protein were enriched by biotin agarose beads. The capture protein was identified by mass spectrometry and the non-specific binding was removed.

The Specific Steps are as Follows:

2.1 Biotin-modified CobB protein was obtained. The CobB sequence with Avi tag was constructed into pET32a vector, and BirA with biotin labeling function was constructed into pET28a vector, and both vectors were transformed into E. coli BL21 (DE3) strain at the same time. The biotin-modified CobB protein was purified by nickel column after IPTG was added when OD600 was about 0.6 and induced overnight at 18° C.

2.2 Preparation of Cell Lysate Samples

E. coli cells and SLIAC (Lys/Arg re-labeled) cells were cultured and lysed by high pressure disruption to obtain cell lysate.

2.3 Capture and Detection of Protein Interactions

The following reaction system was prepared: 1 μM biotinylated CobB protein, 5 μM SAₘ-Pup^{E}, 0.5 μM PafA7_{KR} enzyme, 5 mM ATP, 5 mg of E. coli cell lysate or SILAC cell lysate were added, and buffer (50 mM Tris 8.0, 0.5 M NaCl, 20 mM MgCl₂, 10% (v/v) Glycerol, 10 mM imidazole) was lysed to 5 ml. The system was incubated at 30° C. for 6h, and biotin agarose beads were added for incubation overnight at 4° C. Wash buffer 1 (8M urea, 50 mM Tris 8.0, 200 mM NaCl, 0.2% SDS), Wash Buffer 2 (8M urea, 50 mM Tris 8.0, 200 mM NaCl, 2% SDS), Wash buffer 3 (8M urea, 50 mM Tris 8.0, 200 mM NaCl), Wash buffer 4 (50 mM Tris 8.0, 0.5 mM EDTA, 1 mM DTT), Wash buffer 5 (50 mM NH4HCO₃) were incubated at room temperature for 5 min and centrifuged at 1500 rpm for 4 min. The biotin agarose beads were transferred to a 1.5 mL centrifuge tube, centrifuged in a horizontal centrifuge with 1 mL Wash buffer 5, 1500 rpm for 4 min and washed repeatedly with Wash buffer 5 to obtain the reaction product. Trypsin was lysed and identified by mass spectrometry.

3. Result Analysis

Comparing the CobB interacting proteins obtained by this method with the existing studies, the results are as shown in FIG. 7C. A total of 261 CobB interacting proteins were detected by this method, of which 122 were consistent with the existing studies, 34 were consistent with the existing two studies, and 139 interacting proteins were detected for the first time.

4. Interacting Protein Validation

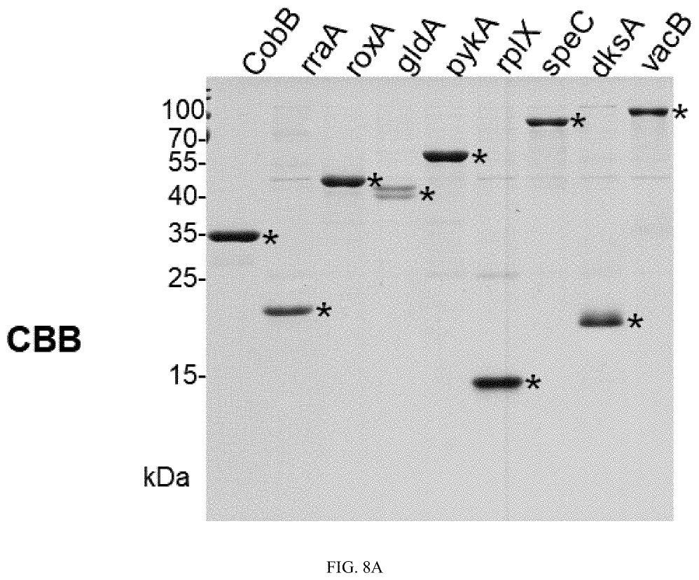

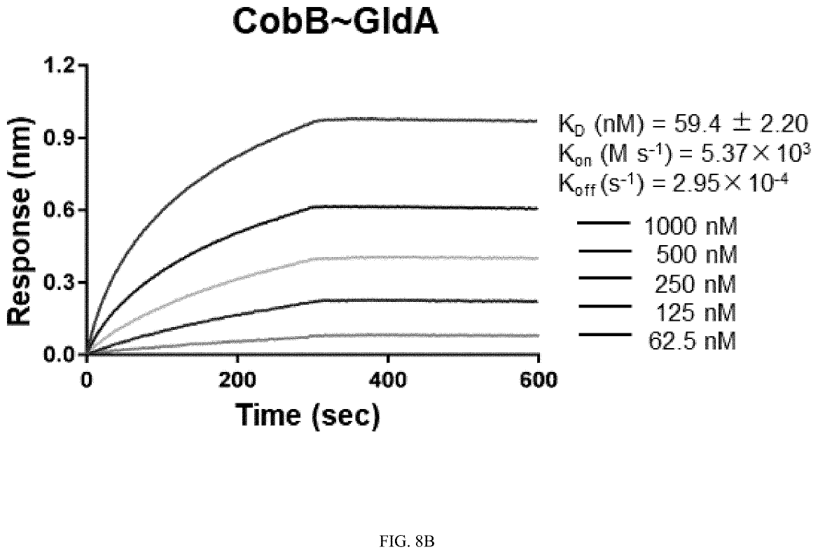

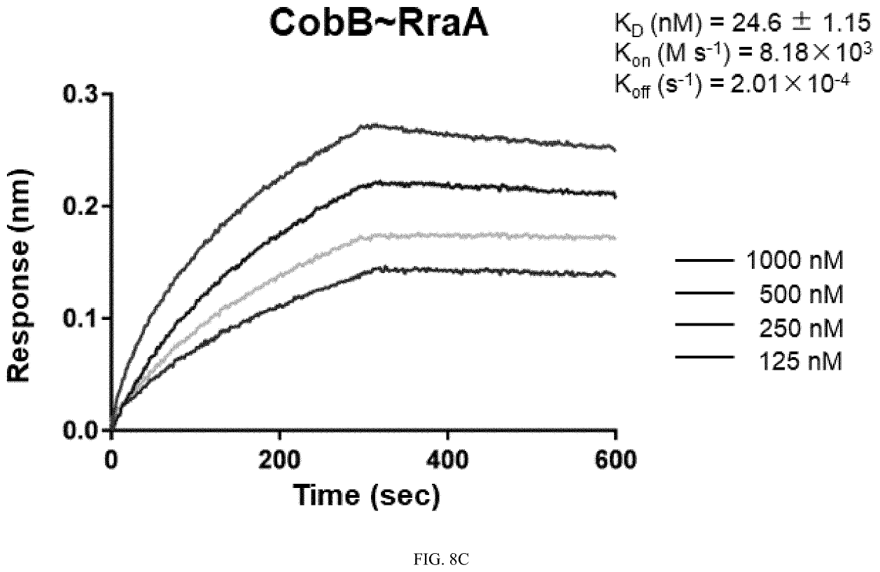

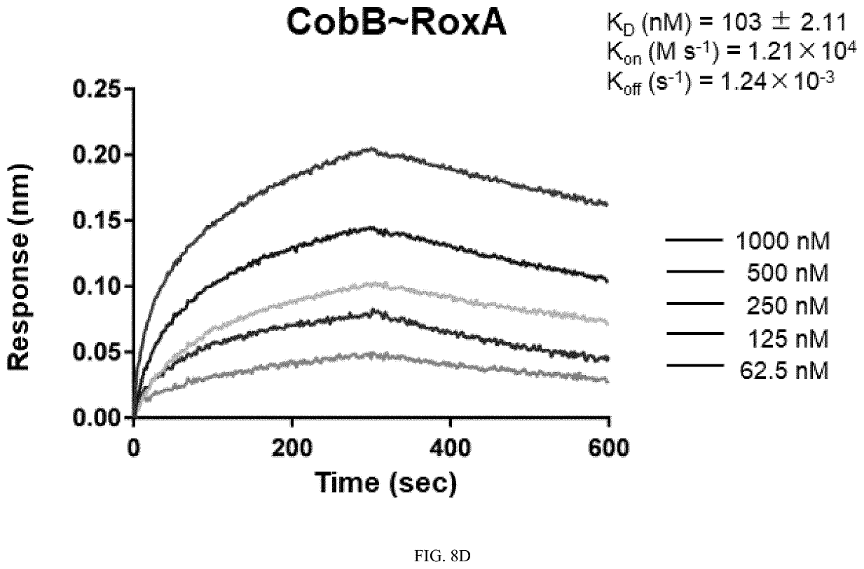

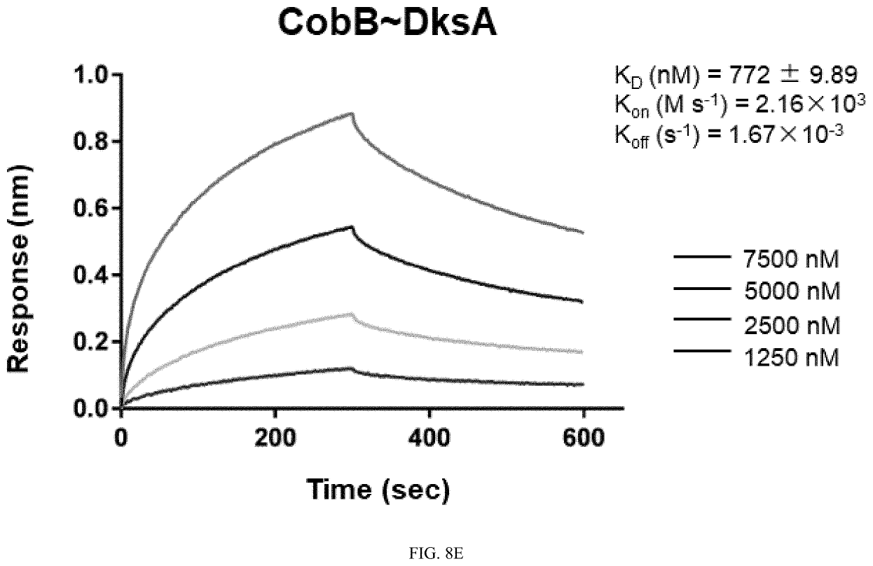

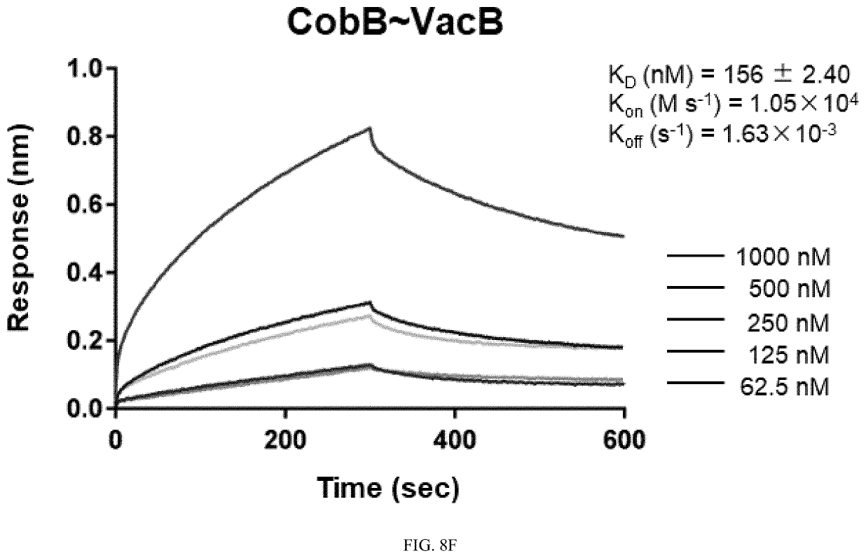

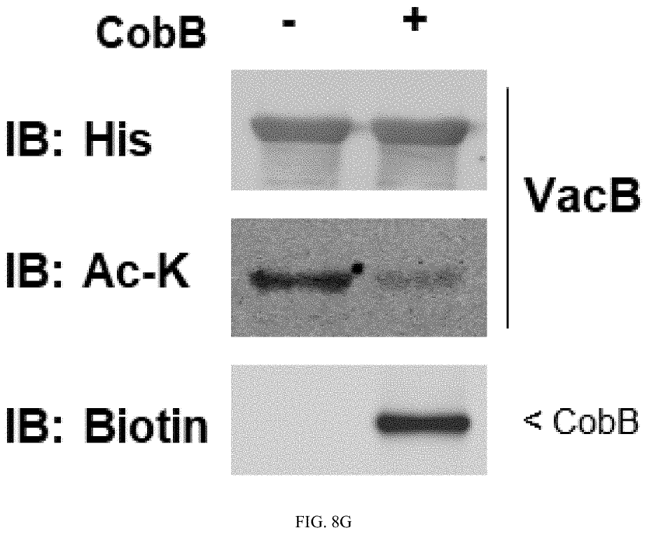

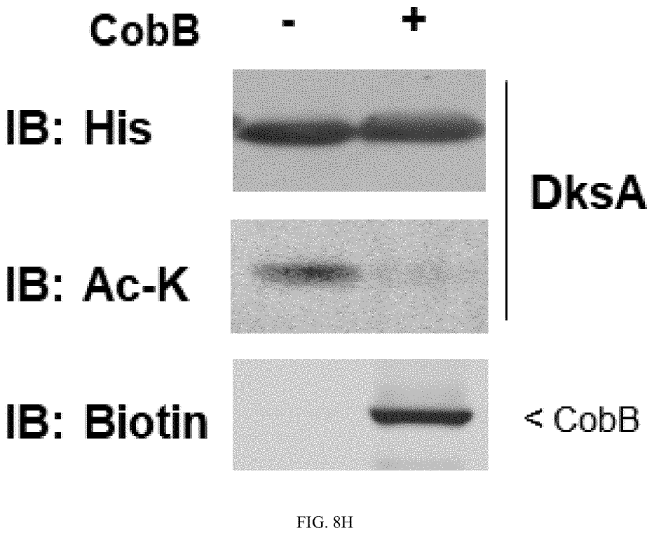

The interacting proteins found were purified and the interaction with CobB was verified. The results of purified proteins are shown in FIG. 8A, and the KD value of the interaction between proteins and CobB detected by BLI is between 25 and 772 nM, as shown in FIG. 8B to f. It shows that this method can detect protein-protein interactions with large affinity range.

CobB has the activity of deacetylase. CobB was co-incubated with VacB and DksA proteins, and the acetylation level of protein was detected by Western blot analysis of acetylation antibody. As shown in FIGS. 8G-8H, the acetylation level of a group of proteins added with CobB decreased obviously, which indicated that CobB played a deacetylation role on VacB and DksA, and functionally proved the interaction between CobB and VacB and DksA.

### Embodiment 6 Detection of Cell Surface Receptor of PD-1 Protein

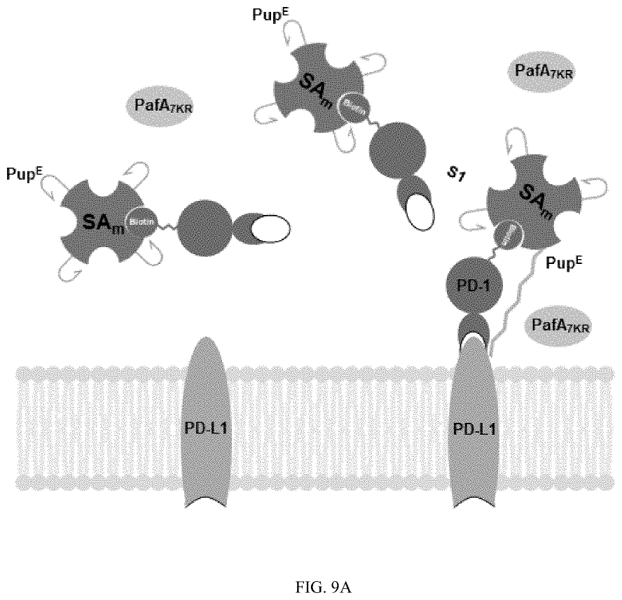

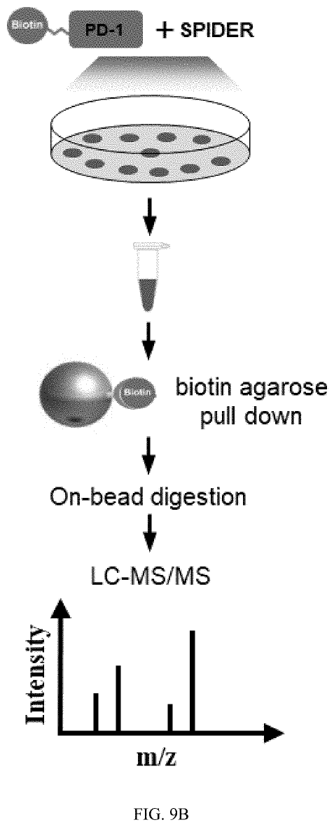

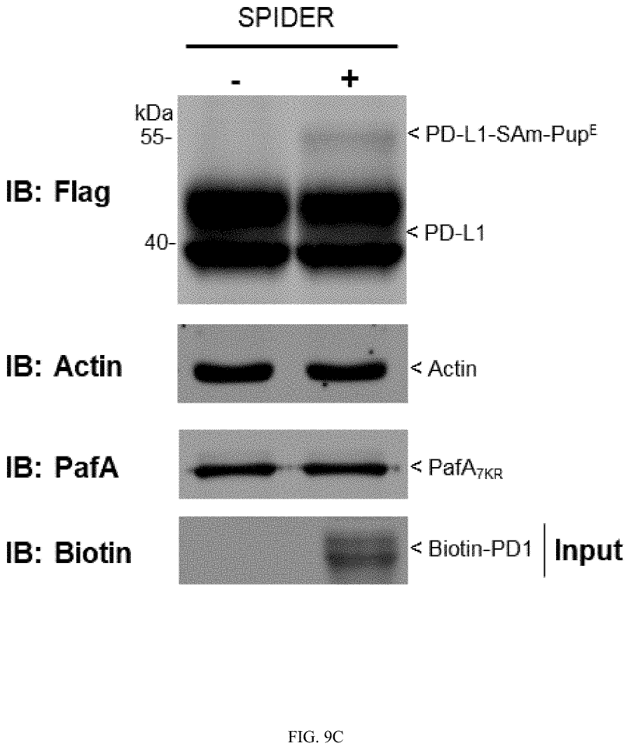

1. The principle of using this method to detect PD-1 protein cell surface receptor is shown in FIG. 9A.

Biotinylated PD-1 protein binds to SAₘ-Pup^{E}. When PD-1 interacts with cell surface receptor, PafA7_{KR} exerts proximity labeling activity to covalently link the C terminal of SAₘ-Pup^{E} to the receptor.

2. The Flow of Detecting the Cell Surface Receptor of PD-1 Protein Using this Method is Shown in FIG. 9B.

Firstly, purified PD-1 protein was constructed and reacted with HEK293T living cells in Petri dish. SAₘ-Pup^{E} and its covalently linked capture protein were enriched by biotin agarose beads, and the capture protein was identified by mass spectrometry.

The specific steps are as follows:

2.1 Construction of PD-1 sequence with Avi tag on N-terminal onto pET32a vector, construction of BirA with biotin-labeled function onto pET28a vector, co-transformation of the two plasmids into E. coli BL21 (DE3) strain. When OD600 was 0.6, IPTG was added and induced overnight at 18° C. The PD-1 protein modified by biotin was purified by nickel column.

2.2 Preparation of HEK293T Cells

The PD-L1 plasmid was transfected into HEK293T cells with Lipofectamine 2000 (ThermoFisher 118668) and cultured for 48 h.

2.3 Capture of Cell Surface Receptors

The following reaction system was prepared: 1 μM biotinylated PD-1 protein, 5 μM SAₘ-Pup^{E}, 0.5 μM PafA7_{KR} enzyme, 5 mM ATP. HEK293T cells overexpressing PD-L1 were added to react in a plate, incubated at 30° C. for 6h, and incubated overnight at 4° C. with biotin agarose beads. Wash buffer 1 (8M urea, 50 mM Tris 8.0, 200 mM NaCl, 0.2% SDS), Wash Buffer 2 (8M urea, 50 mM Tris 8.0, 200 mM NaCl, 2% SDS), Wash buffer 3 (8M urea, 50 mM Tris 8.0, 200 mM NaCl), Wash buffer 4 (50 mM Tris 8.0, 0.5 mM EDTA, 1 mM DTT), Wash buffer 5 (50 mM NH4HCO3) were incubated at room temperature for 5 min and centrifuged at 1500 rpm for 4 min. The biotin agarose beads were transferred to a 1.5 mL centrifuge tube, centrifuged in a horizontal centrifuge with 1 mL Wash buffer 5, 1500 rpm for 4 min and washed repeatedly with Wash buffer 5 to obtain the reaction product. Trypsin was lysed and identified by mass spectrometry.

3. Verification of Interaction Between PD-1 and PD-L1

The invention is used to verify the interaction between PD-1 and PD-L1. If there is interaction, PafA7_{KR} enzyme covalently connects the C terminal of SAₘ-Pup^{E} to PD-L1. As shown in FIG. 9C, when PD-L1 overexpressed cell lysate was co-incubated with PD-1, Western blot showed an upward shift band above PD-L1, which proved the interaction between PD-1 and PD-L1.

### Embodiment 7 Detection of Interacting Proteins of SARS-CoV-2 Proteins

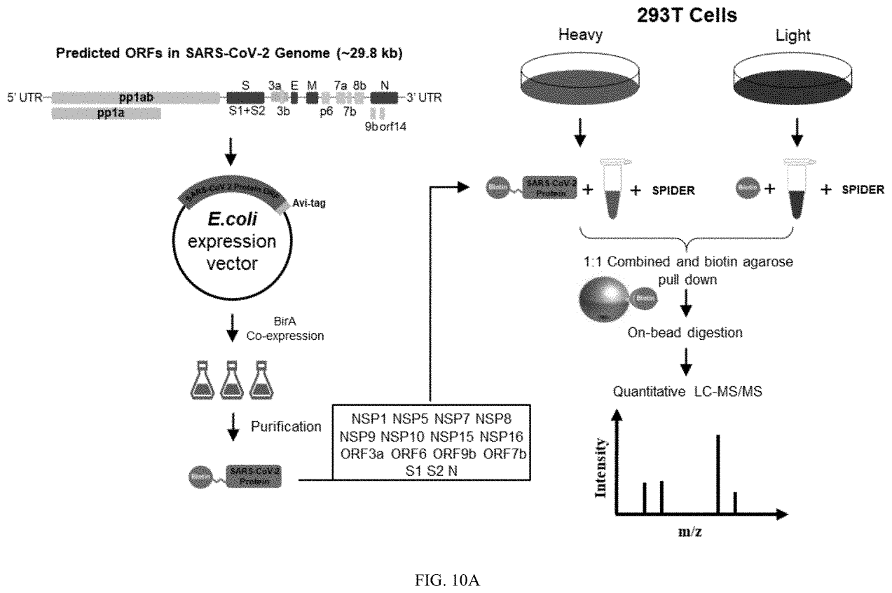

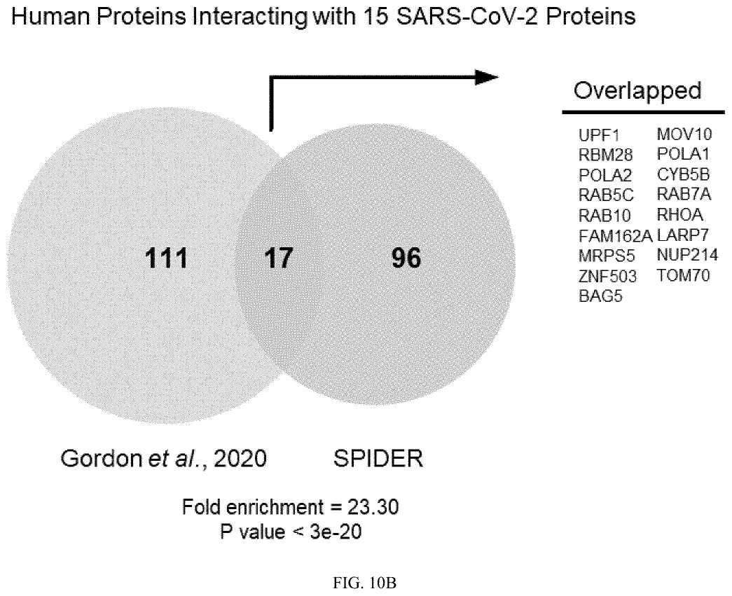

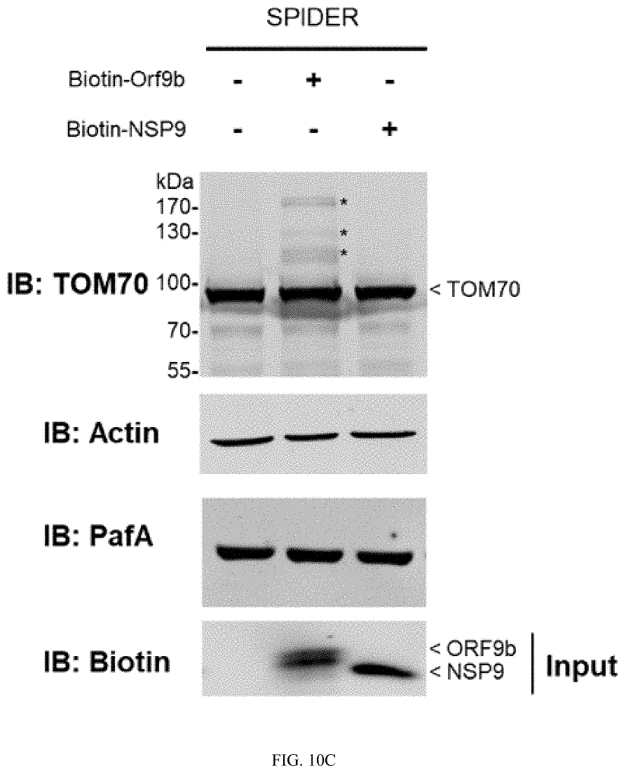

1. The flow of using this method to detect the interaction protein of some SARS-CoV-2 proteins is shown in FIG. 10A.

Biotin modified SARS-CoV-2 protein was obtained. SARS-CoV-2 protein sequence with Avi tag was constructed into pET32a vector, and BirA with biotin labeling function was constructed into pET28a vector, and 2 plasmids were co-transformed into E. coli BL21 (DE3) strain. The biotin-modified SARS-CoV-2 protein was purified by nickel column after IPTG was added when OD600 was about 0.6 and induced overnight at 18° C.

2. Preparation of Samples to be Tested

HEK293T normal cells and SLIAC (Lys/Arg relabeled) cells were cultured and lysed with Thermo Fisher 78501 M-PER® Mammalian Protein Extraction to obtain cell lysates.

3. Capture and Detection of Protein-Protein Interactions

The reaction system was prepared as follows: 1 μM biotinylated bait protein, 5 μM SAₘ-Pup^{E}, 0.5 μM PafA, 5 mM ATP, 5 mg HEK293T cell lysate or SILAC cell lysate was added, and the volume was supplemented to 5 ml with M-PER lysate. The system was incubated at 30° C. for 6h, and biotin agarose beads were added for incubation overnight at 4° C. Wash buffer 1 (8M urea, 50 mM Tris 8.0, 200 mM NaCl, 0.2% SDS), Wash Buffer 2 (8M urea, 50 mM Tris 8.0, 200 mM NaCl, 2% SDS), Wash buffer 3 (8M urea, 50 mM Tris 8.0, 200 mM NaCl), Wash buffer 4 (50 mM Tris 8.0, 0.5 mM EDTA, 1 mM DTT), Wash buffer 5 (50 mM NH₄HCO₃) were incubated at room temperature for 5 min and centrifuged at 1500 rpm for 4 min. The biotin agarose beads were transferred to a 1.5 mL centrifuge tube, centrifuged in a horizontal centrifuge with 1 mL Wash buffer 5, 1500 rpm for 4 min and washed repeatedly with Wash buffer 5 to obtain the reaction product. Trypsin was lysed and identified by mass spectrometry.

4. Result Reading

Comparing the SARS-CoV-2 protein interacting proteins obtained by this method with the existing methods, the results are as shown in FIG. 10B. A total of 113 SARS-CoV-2 protein interacting proteins were detected by this method, among which 96 new interacting proteins were detected, and 17 were consistent with the existing studies.

5. Interacting Protein Validation

The purified biotinylated ORF9b was co-incubated with SAₘ-Pup^{E}, cell lysate overexpressing TOM70, PafA and ATP, and the bands of covalent linkage between TOM70 and SAₘ-Pup^{E} monomer were detected, which indicated protein-protein interaction. As shown in FIG. 10C, ORF9b interacted with TOM70, while Nsp9, another protein of SARS-CoV-2 in control group, did not interact with TOM70.

### Embodiment 8 Identification of Interacting Proteins of Biotinylated m6A RNA

1. Obtain Biotin-Modified RNA.

The 5′-terminal biotin-modified m6A RNA (biotinylated m6A RNA) was synthesized by Nanjing Kingsley Company, and the RNA sequence was CGUCUCGGCUCGGCUGCU (SEQ ID NO: 8).

2. Prepare Samples to be Tested

HEK293T normal cells and SLIAC (Lys/Arg relabeled) cells were cultured and lysed with Thermo Fisher 78501 M-PER® Mammalian Protein Extraction to obtain cell lysates.

3. Capture and Detection of m6A RNA Interacting Proteins

The reaction system was prepared as follows: 1 μM biotinylated m6A RNA, 5 μM SAₘ-Pup^{E}, 0.5 μM PafA7_{KR} enzyme, 5 mM ATP, 5 mg HEK293T cell lysate or SILAC cell lysate were added, and buffer (50 mM Tris 8.0, 0.5 M NaCl, 20 mM MgCl₂, 10% (v/v) Glycerol, 10 mM imidazole) was lysed to 5 ml. The system was incubated at 30° C. for 6h, and biotin agarose beads were added for incubation overnight at 4° C. Wash buffer 1 (8M urea, 50 mM Tris 8.0, 200 mM NaCl, 0.2% SDS), Wash Buffer 2 (8M urea, 50 mM Tris 8.0, 200 mM NaCl, 2% SDS), Wash buffer 3 (8M urea, 50 mM Tris 8.0, 200 mM NaCl), Wash buffer 4 (50 mM Tris 8.0, 0.5 mM EDTA, 1 mM DTT), Wash buffer 5 (50 mM NH₄HCO₃) were incubated at room temperature for 5 min and centrifuged at 1500 rpm for 4 min. The biotin agarose beads were transferred to a 1.5 mL centrifuge tube, centrifuged in a horizontal centrifuge with 1 mL Wash buffer 5, 1500 rpm for 4 min and washed repeatedly with Wash buffer 5 to obtain the reaction product. Trypsin was lysed and identified by mass spectrometry. Three credible m6A binding proteins, YTDHF1, YTDHF2 and YTDHF3, were obtained by mass spectrometry.

### Embodiment 9: Verification of Interaction Between Biotinylated m6A RNA and YTDHF1, YTDHF2, YTDHF3 Proteins

1. Obtain Biotin-Modified m6A RNA

The 5′-terminal biotin-modified m6A RNA (biotinylated m6A RNA) was synthesized by Nanjing Kingsley Company, and the RNA sequence was CGUCUCGGCUCGGCUGCU (SEQ ID NO: 8). The cell lysates overexpressing YTDHF1, YTDHF2 and YTDHF3 proteins were obtained.

2. Prepare Samples to be Verified

The sequences of YTDHF1, YTDHF2 and YTDHF3 were linked with GFP tags, which were used for Western blot detection. The sequences of YTDHF1-GFP, YTDHF2-GFP and YTDHF3-GFP were constructed on pCDNA3.1 vector and transfected into HEK293T cells with Lipofectamine 2000 (ThermoFisher 118668). After 48h of culture, the lysates overexpressing YTDHF1, YTDHF2 and YTDHF3 were extracted.

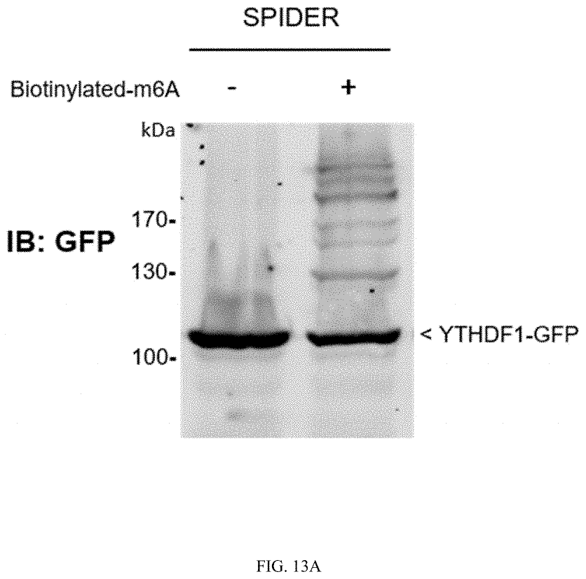

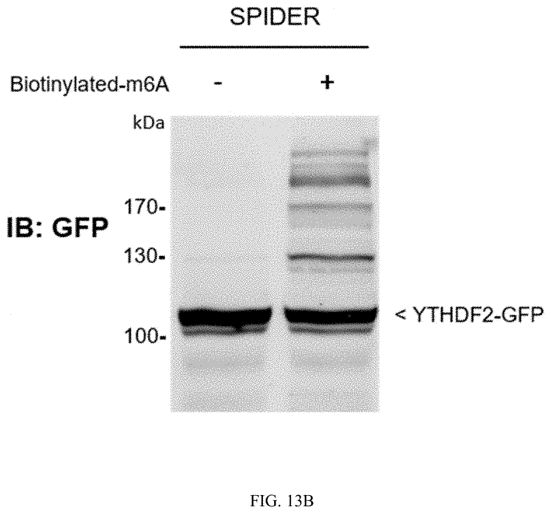

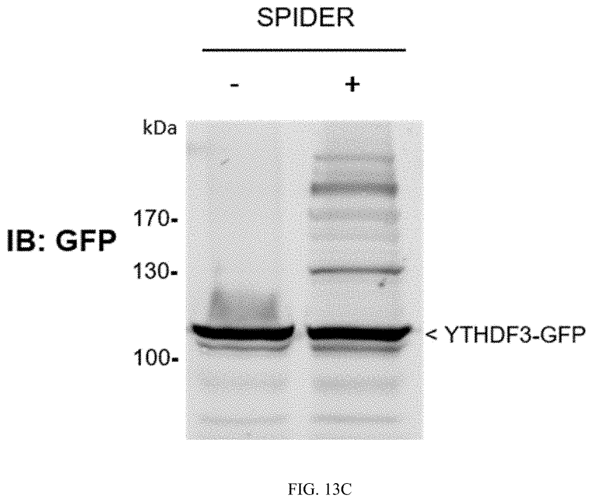

3. Verify the Interaction of Biotinylated m6A RNA with YTDHF1, YTDHF2, YTDHF3 Proteins, as Shown in FIGS. 13A-13C.

The cells overexpressing YTDHF1, YTDHF2 and YTDHF3 were mixed with biotinylated m6A RNA (0.5 μM) and SAₘ-Pup^{E}, then PafA7_{KR} (10 mM) and ATP (5 mM) were added into the system, then incubated at 30° C. for 4-6h. Immunoblot analysis of GFP antibody was used. If the protein YTDHF1, YTDHF2, YTDHF3 interacted with biotinylated m6A RNA, the protein-SAₘ-Pup^{E} complex bands (>120 kDa) could be detected; If the proteins YTDHF1, YTDHF2, YTDHF3 did not interact with biotinylated m6A RNA, only YTDHF1, YTDHF2, YTDHF3 bands (about 100 kDa) could be detected. As shown in FIGS. 13A-13C, the presence of complex bands only in the presence of biotinylated m6A RNA indicates that the present invention is capable of specifically verifying the interaction of biotinylated RNA with proteins.

This example also verified that the streptavidin-Pup tetramer protein prepared using the Pup (E) of example 1 (the preparation method is the same as that of example 2, except that Pup^{E} is replaced by Pup (E)) was used to verify the interaction of biotinylated m6A RNA with YTDHF1, YTDHF2, YTDHF3 proteins, and the results were similar to those of FIGS. 13A-13C.

This example also verified that SAₘ-Pup^{E-1} and SAₘ-Pup^{E-2} described in Example 2 were used to verify the interaction between biotinylated m6A RNA and YTDHF1, YTDHF2 and YTDHF3 proteins, and the results were similar to those of FIGS. 13A-13C.

### Embodiment 10 Identification of Interacting Proteins of Biotinylated DNA

In this example, four segments of biotinylated DNA are used, and the interacting proteins are identified after mixing them. The target sequences of the four segments of biotinylated DNA are CGGCAGATGCATAAAGGTG (SEQ ID NO: 9), CACCTTTTTTATGCATCTGCCG (SEQ ID NO: 10), CCTTTTTTATGCAAAT (SEQ ID NO: 11) and ATATGCAAATT (SEQ ID NO: 12).

1. Obtain Biotin-Modified DNA

Nanjingjinsirui Science & Technology Biology Corp. synthesized the above four 5′-end biotin modified DNA target sequences and their corresponding four complementary sequences, DNA target sequence (DNA oligo A) and its corresponding complementary sequence (DNA oligo B) were prepared into 5004 with ultrapure water, respectively, and the following reaction systems were set up: Nuclease-Free Water 40 μL, Annealing Buffer (5×) 20 μL, DNA oligo A (50 μM) 20 μL, DNA oligo B (50 μM) 20 μL. After the above systems were mixed evenly, they were placed in PCR to perform annealing reaction: 95° C. for 2 min, and dropped 0.1° C. every 8 seconds to 25° C., and then double-stranded target DNA (i.e. biotinylated DNA) was obtained.

2. Obtain the Sample to be Tested

Mouse cells were cultured and lysed using Thermo Fisher 78501 M-PER® Mammalian Protein Extraction to obtain cell lysate.

3. Capture and Detection of Biotinylated DNA Interacting Proteins

The following reaction system was prepared: 1 μM mixed biotinylated DNA, 5 μM SAₘ-Pup^{E}, 0.504 PafA7_{KR} enzyme, 5 mM ATP, 5 mg HEK293T cell lysate or SILAC cell lysate, and 5 ml buffer (50 mM Tris 8.0, 0.5 M NaCl, 20 mM MgCl₂, 10% (v/v) Glycerol, 10 mM imidazole). The system was incubated at 30° C. for 6h, and biotin agarose beads were added for incubation overnight at 4° C. Wash buffer 1 (8M urea, 50 mM Tris 8.0, 200 mM NaCl, 0.2% SDS), Wash buffer 2 (8M urea, 50 mM Tris 8.0, 200 mM NaCl, 2% SDS), Wash buffer 3 (8M urea, 50 mM Tris 8.0, 200 mM NaCl), Wash buffer 4 (50 mM Tris 8.0, 0.5 mM EDTA, 1 mM DTT) and Wash buffer 5 (50 mM NH₄HCO₃) were incubated at room temperature for 5 min, and centrifuged at 1500 rpm for 4 min to remove supernatant. The biotin agarose beads were transferred to a 1.5 mL centrifuge tube, centrifuged in a horizontal centrifuge with 1 mL Wash buffer 5, 1500 rpm for 4 min and washed repeatedly with Wash buffer 5 to obtain the reaction product. Trypsin was lysed and identified by mass spectrometry. According to the results of mass spectrometry, several credible biotin DNA binding proteins were obtained, such as Sox2, HNRNPAB, Sub1, Arid 3a, etc.

### Embodiment 11 Verification of Interaction Between Biotinylated DNA and EthR Protein

1. Obtain Biotin-Modified DNA

Nanjingjinsirui Science & Technology Biology Corp. synthesized the 5′-terminal biotin modified DNA target sequence and its complementary sequence. The DNA target sequence is: CATGGATCCACCGTAATGTCGAGGCCGTCAACGAGATGTCGACACTATCGACACGT AGTAAGCTGCCAGATGAGACAAA (SEQ ID NO: 13). DNA target sequence (DNA oligo A) and its corresponding complementary sequence (DNA oligo B) were prepared into 50 μM with ultrapure water, respectively, and the following reaction systems were set up: Nuclease-Free Water 40 μL, Annealing Buffer (5×) 20 μL, DNA oligo A (50 μM) 20 μL, DNA oligo B (50 μM) 20 μL. After the above systems were mixed evenly, they were placed in PCR to perform annealing reaction: 95° C. for 2 min, and dropped 0.1° C. every 8 seconds to 25° C., and then double-stranded target DNA (i.e. biotinylated DNA) was obtained.

2. Obtain DNA Binding Protein EthR

EthR-Flag-His sequence was obtained by linking 6×His and Flag tags to the sequence encoding EthR protein, in which 6×His tags were used for protein purification and Flag tags were used for Western blot detection. EthR-Flag-His sequence was constructed on pET28a vector and transformed into E. coli BL21 (DE3) strain. When OD600≈0.6, IPTG was added into 1 L bacterial solution cultured, and induced overnight at 18° C. EthR protein was purified by nickel column.

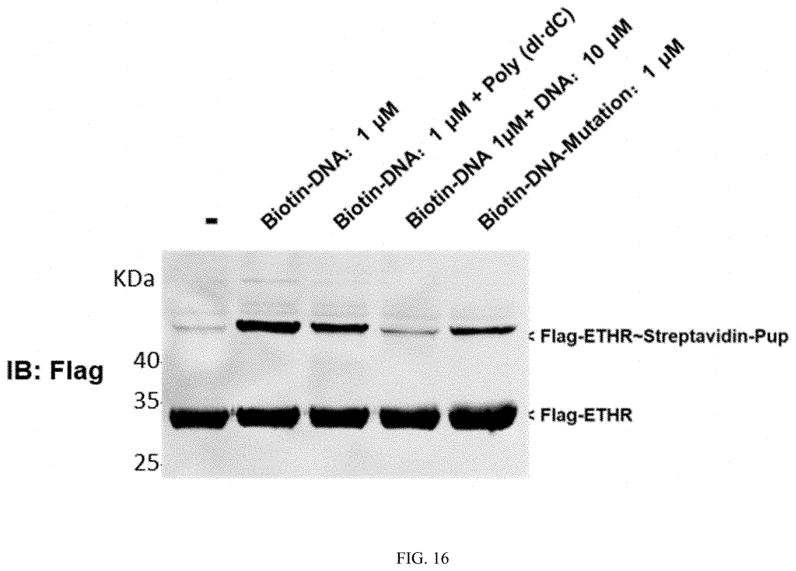

3. Verify the Interaction Between Biotinylated DNA and EthR, as Shown in FIG. 16.

In order to verify that interaction between specifically capture biotinylated DNA and EthR protein, Using different types of DNA molecules to react with EthR protein, The biotinylated DNA molecule is used as the experimental group, poly dIdC molecule can reduce the non-specific binding between DNA and protein, high concentration of DNA molecules without biotin modification and with the same sequence are used to compete for binding to low concentration of biotinylated DNA, and mutated biotinylated DNA (sequence: CATGGATCCACCGCTATCAACGTAATGCCGTCAACAAGATAAGCCCCCTATCGACAC GTAGTAAGCTGCCAGATGACAAAGCCID, SEQ ID NO: 14) is used to verify the specificity of DNA sequence. Biotinylated DNA (1 μM), a mixture of biotinylated DNA (1 μM) and poly dI dC, a mixture of biotinylated DNA (1 μM) and identical DNA without biotin modification (10 μM), and mutated biotinylated DNA (1 μM) were added to several systems. Different types of EthR-binding DNA fragments were mixed with EthR protein (0.2 μM) and SAₘ-Pup^{E}. PafA7_{KR} (10 mM) and ATP (5 mM) were added into the system, and incubated at 30° C. for 4-6h. Flag antibody was used for Western blot analysis. If the protein EthR interacted with DNA, the complex band (about 50 kDa) between EthR and SAₘ-Pup^{E} monomer was detected; If there was no interaction between the protein EthR and DNA, an EthR band (about 32 kDa) was detected. As shown in FIG. 16, the complex bands are thickest only in the presence of biotinylated DNA, indicating that the present invention can specifically verify the interaction between biotinylated DNA and protein.

### Embodiment 12 Verification of Specificity of DNA-RutR Interaction

1. Obtain Biotin-Modified DNA

Nanjingjinsirui Science & Technology Biology Corp. synthesized the 5′-end biotin modified DNA target sequence and its complementary sequence. 2 DNA target sequences are TTGACCACATGGACCAAACAGTCTG (SEQ ID NO: 15, corresponding to the DNA sequence of biotin-D1 or D1) and TTGACCACATAGACCGACTGGTCTA (SEQ ID NO: 16, corresponding to the DNA sequence of biotin-D2 or D2). DNA target sequence (DNA oligo A) and its corresponding complementary sequence (DNA oligo B) were prepared into 50 μM with ultrapure water, respectively, and the following reaction systems were set up: Nuclease-Free Water 40 μL, Annealing Buffer (5×) 20 μL, DNA oligo A (50 μM) 20 μL, DNA oligo B (50 μM) 20 μL. After the above systems were mixed evenly, they were placed in PCR to perform annealing reaction: 95° C. for 2 min, and dropped 0.1° C. every 8 seconds to 25° C., and then double-stranded target DNA (i.e. biotinylated DNA) was obtained.

2. Obtain DNA Binding Protein RutR

RutR-Flag-His sequence was obtained by linking 6×His and Flag tags to the sequence encoding RutR protein, in which 6×His tags were used for protein purification and Flag tags were used for Western blot detection. The RutR-Flag-His sequence was constructed on pET28a vector and transformed into E. coli BL21 (DE3) strain. The RutR protein was obtained by adding IPTG at 18° C. overnight when OD600 was about 0.6 and purified by nickel column.

3. Verify the Interaction Between Biotinylated DNA and RutR, as Shown in FIG. 17.

In order to verify that the invention can specifically capture the interaction between biotinylated DNA and RutR protein, different types of DNA molecules are used to react with RutR protein: biotinylated DNA (biotin-D1, biotin-D2), DNA without biotin modification and consistent sequence (D1, D2), irrelevant sequence D3 (the sequence of D3 is CAACCATGAGTCATAC, SEQ ID NO: 17) and biotin labeled D3 (biotin-D3); biotin-D1 (1 μM), biotin-D1 (1 μM) and D1 (10 μM), biotin-D2 (1 μM), biotin-D2 (1 μM) and D2 (10 μM), biotin-D3 (1 μM), biotin-D3 (1 μM) and D3 (10 μM) were added to different reaction systems (as shown in FIG. 17). Several DNA fragments were well mixed with RutR protein (0.2 μM) and SAₘ-Pup^{E}. PafA7_{KR} (10 mM) and ATP (5 mM) were added into the system, and incubated at 30° C. for 4-6 h. Flag antibody was used for Western blot analysis. If the protein RutR interacted with DNA, the complex band of RutR and SAₘ-Pup^{E} monomer was detected (about 52 kDa); If there was no interaction between RutR and DNA, RutR bands (about 30 kDa) were detected. As shown in FIG. 17, the complex bands of RutR and SAₘ-Pup^{E} monomer appear only in the presence of biotinylated DNA (biotin-D1, biotin-D2), indicating that the present invention can specifically verify the interaction between biotinylated DNA and protein.

### Example 13 Verification of Interaction Between Biotinylated DNA and GCN4

1. Obtain Biotin-Modified DNA

Nanjingjinsirui Science & Technology Biology Corp. synthesized the 5′-terminal biotin modified DNA target sequence and its complementary sequence, and the DNA target sequence was CAACCCATGAGTCATAC (SEQ ID NO: 17). DNA target sequence (DNA oligo A) and its corresponding complementary sequence (DNA oligo B) were prepared into 50 μM with ultrapure water, respectively, and the following reaction systems were set up: Nuclease-Free Water 40 μL, Annealing Buffer (5×) 20 μL, DNA oligo A (50 μM) 20 μL, DNA oligo B (50 μM) 20 μL. After the above systems were mixed evenly, they were placed in PCR to perform annealing reaction: 95° C. for 2 min, and dropped 0.1° C. every 8 seconds to 25° C., and then double-stranded target DNA (i.e. biotinylated DNA) was obtained.

2. Obtain DNA Binding Protein GCN4

GCN4-Flag-His sequence was obtained by linking 6×His and Flag tags to the sequence encoding GCN4 protein, in which 6×His tags were used for protein purification and Flag tags were used for Western blot detection. The GCN4-Flag-His sequence was constructed on pET28a vector and transformed into E. coli BL21 (DE3) strain. When OD600 was 0.6, IPTG was added and induced overnight at 18° C., and GCN4 protein was purified by nickel column.

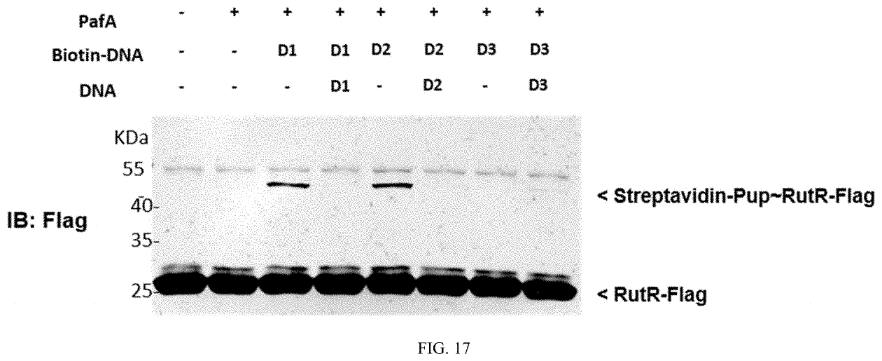

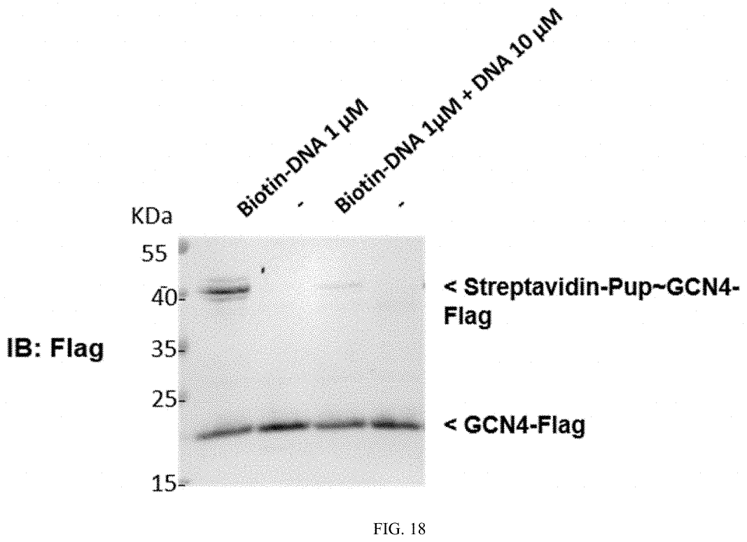

3. Verify the Interaction Between Biotinylated DNA and GCN4, as Shown in FIG. 18.

The biotinylated DNA molecule (104) was fully mixed with GCN4 protein (0.2 μM) and SAₘ-Pup^{E}, and a high concentration of DNA (10 μM) without biotin modification and with consistent sequence was added to the other system to verify that SPIDER technology specifically captured the interaction between biotinylated DNA and GCN4 protein. PafA7_{KR} (10 mM) and ATP (5 mM) were added into the system, and then incubated at 30° C. for 4-6 h. Flag antibody was used for Western blot analysis. If the protein GCN4 interacted with DNA, the complex band (about 40 kDa) between GCN4 and SAₘ-Pup^{E} monomer was detected; If there was no interaction between GCN4 and DNA, GCN4 bands (about 20 kDa) were detected. As shown in FIG. 18, the complex bands of GCN4 and SAₘ-Pup^{E} monomer appear only in the presence of biotinylated DNA, indicating that the present invention can specifically verify the interaction between biotinylated DNA and protein.

### Embodiment 14 Identification of Interacting Proteins of Lenalidomide Small Molecule

1. Obtain Biotin Modified Lenalidomide (Lenalidomide) Small Molecule

Biotin modified lenalidomide molecule was presented by Dong Jiajia, a teacher from Shanghai Institute of Organic Chemistry, Chinese Academy of Sciences. lenalidomide is a conventional small molecule with a size of 259.261 Da.

2. Obtain the Sample to be Tested

HEK293T cells were cultured and lysed with Thermo Fisher 78501 M-PER® Mammalian Protein Extraction to obtain cell lysate.

3. Capture and Detection of Biotinylated Lenalidomide Interacting Protein

The reaction system was prepared as follows: 1 μM mixed biotinylated lenalidomide, 5 μM SAₘ-Pup^{E}, 0.5 μM PafA7_{KR} enzyme, 5 mM ATP, 5 mg of HEK293T cell lysate or SILAC cell lysate was added, and buffer (50 mM Tris 8.0, 0.5 M NaCl, 20 mM MgCl2, 10% (v/v) Glycerol, 10 mM imidazole) was completed to 5 ml. The system was incubated at 30° C. for 6h, and biotin agarose beads were added for incubation overnight at 4° C. Wash buffer 1 (8M urea, 50 mM Tris 8.0, 200 mM NaCl, 0.2% SDS), Wash buffer 2 (8M urea, 50 mM Tris 8.0, 200 mM NaCl, 2% SDS), Wash buffer 3 (8M urea, 50 mM Tris 8.0, 200 mM NaCl), Wash buffer 4 (50 mM Tris 8.0, 0.5 mM EDTA, 1 mM DTT) and Wash buffer 5 (50 mM NH4HCO3) were incubated at room temperature for 5 min, and centrifuged at 1500 rpm for 4 min to remove supernatant. The biotin agarose beads were transferred to a 1.5 mL centrifuge tube, centrifuged in a horizontal centrifuge with 1 mL Wash buffer 5, 1500 rpm for 4 min and washed repeatedly with Wash buffer 5 to obtain the reaction product. Trypsin was lysed and identified by mass spectrometry. According to the results of mass spectrometry, several credible biotin lenalidomide binding proteins were obtained, such as PRDX2, ADD3, TRIM25, PSMEL PHB, WDR18, HK2 and so on.

### Embodiment 15 Verification of the Interaction Between c-Di-GMP Small Molecule and ETHR Protein

1. Obtain Biotin Modified c-Di-GMP

Biotin-modified c-di-GMP was purchased from Biolog Company under item number B098-005 and molecular size 1172 Da.

2. Obtain the Protein to be Verified

ETHR-Flag-His sequence was obtained by linking the sequence encoding ETHR protein with 6×His tag and Flag tag respectively, in which 6×His tag was used for protein purification and Flag tag was used for Western blot detection. The ETHR-Flag-His sequence was constructed on pET28a vector and transformed into E. coli BL21 (DE3) strain. When OD600 was 0.6, IPTG was added and induced overnight at 18° C. ETHR protein was purified by nickel column.

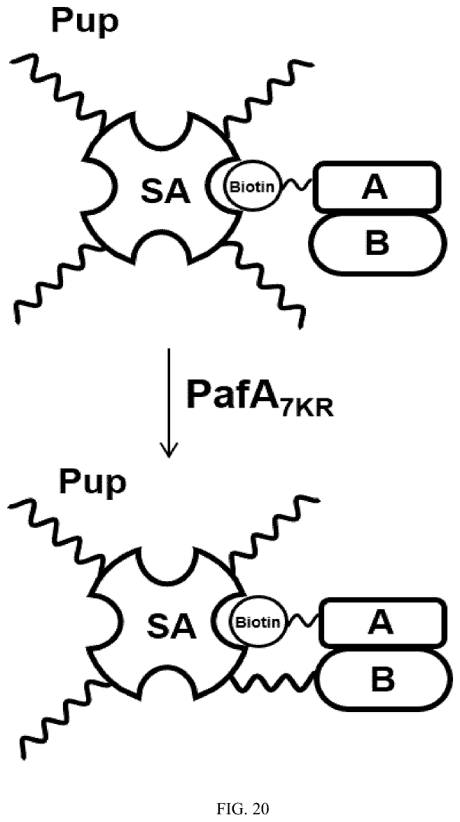

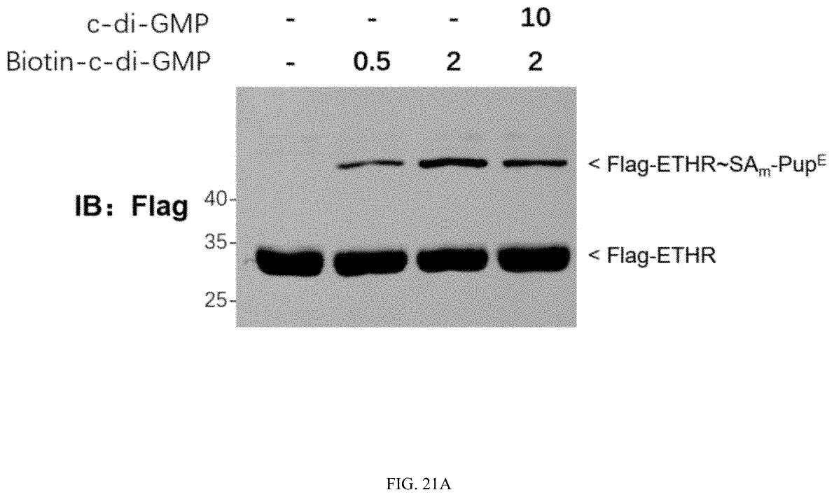

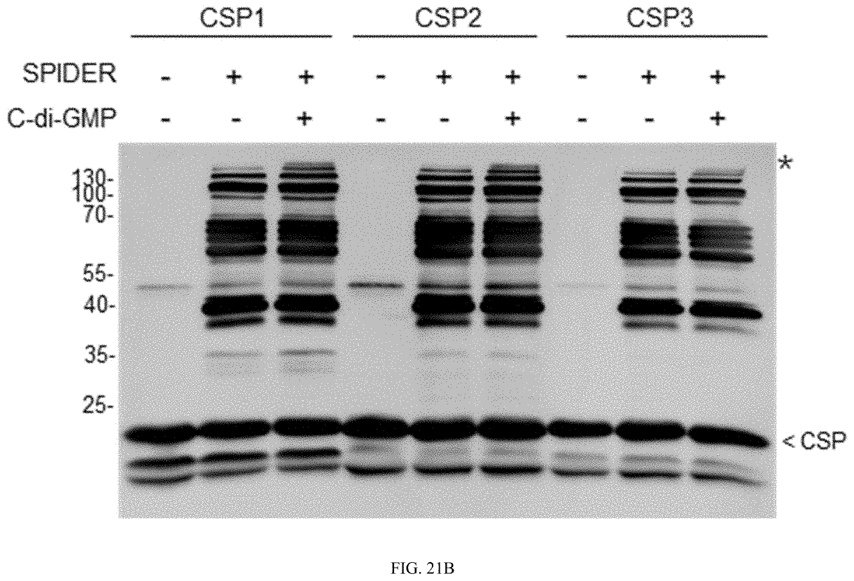

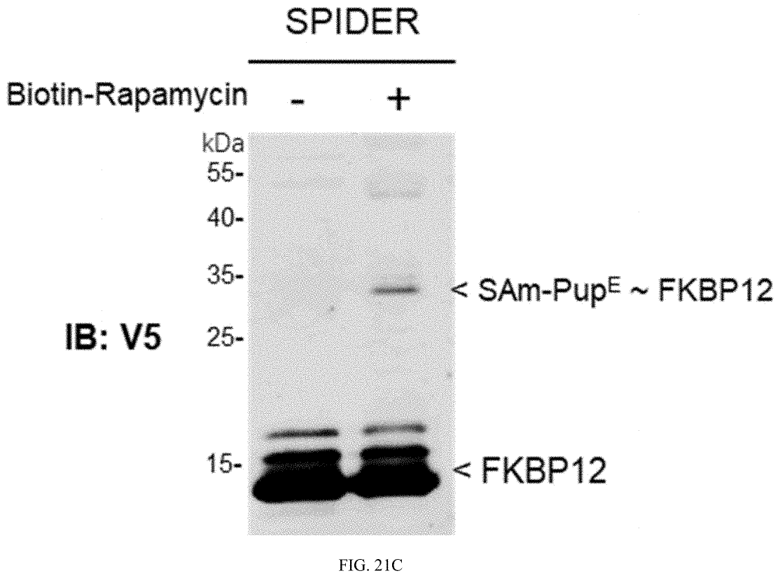

3. Verify the Interaction of c-Di-GMP Small Molecules with ETHR Protein at Different Concentrations, as Shown in FIG. 21A.

The biotinylated small molecule c-di-GMP (Biotin-c-di-GMP) was mixed with ETHR protein and SAₘ-Pup^{E}. The concentration gradients of biotinylated small molecule c-di-GMP are 0, 0.5 μM and 2 μM. PafA 7_{KR} (1 μM) and ATP (10 mM) were added to each concentration system, and then incubated at 30° C. for 4-6 h. Flag antibody was used for Western blot analysis. If the decoy molecule c-di-GMP did not interact with the protein ETHR, only the ETHR protein band (about 32 KDa) was detected; If the small molecule c-di-GMP interacted with the protein, the complex band of ETHR and SAₘ-Pup^{E} monomer (about 50 KDa) was detected. As shown in FIG. 21A, as the concentration of Biotin-C-di-GMP increases, the detected complex bands become thicker, indicating that the present invention verifies that protein interactions are in a concentration-dependent manner. In the fourth reaction system, excessive non-biotinylated c-di-GMP (i.e. c-di-GMP in FIG. 21A) was added to compete for binding biotinylated c-di-GMP, and the complex bands were basically unchanged, indicating that the system specifically bound its interacting proteins through biotinylated c-di-GMP.

### Embodiment 16: Verification of Interaction Between c-Di-GMP Small Molecule and CSP Series Short Peptides

1. Obtain Biotin Modified c-Di-GMP

Biotin-modified c-di-GMP was purchased from Biolog Company under item number B098-005.

2. Obtain the Protein to be Verified

The N-terminal sequences encoding CSP1, CSP2 and CSP3 were all labeled with Flag tags and constructed on PET32a vector, which was fused with thioredoxin for expression. The recombinant vector was transformed into E. coli BL21 (DE3) strain. The CSP1, CSP2 and CSP3 proteins were obtained by adding IPTG and inducing at 37° C. for 4h when OD600 was about 0.6 in 1 L of bacteria medium. The CSP1, CSP2 and CSP3 proteins were purified by nickel column.

CSP series peptide sequences were:

(SEQ ID NO: 18)
CSP1: GGSGDRRRFNSADYKGPRRRKAD
(SEQ ID NO: 19)
CSP2: GGSGDRRFNSADYKGPRRRKAD
(SEQ ID NO: 20)
CSP3: GGSGDRRRFNSADYKAPRRRKAD

3. Verify the Interaction Between c-Di-GMP Small Molecules and CSP Series Proteins, as Shown in FIG. 21B.

The biotinylated small molecule c-di-GMP (2 μM) was mixed with CSP series proteins (5 μM) and SAₘ-Pup^{E} and incubated at 30° C. for 20 min. After that, PafA7_{KR} (1 μM) and ATP (10 mM) were added into the system and incubated at 30° C. for 6 h. Flag antibody was used for Western blot analysis. Compared with c-di-GMP without biotinylation, the CSP series protein bands in the experimental group migrated significantly. The results showed that the system was connected with the whole system by biotinylated c-di-GMP.

### Embodiment 17: Verification of the Interaction Between Rapamycin Small Molecule and FKBP12 Protein

1. Obtain Biotin Modified Rapamycin

Biotin modified Rapamycin was presented by Dang Yongjun, a teacher from School of Basic Medicine, Fudan University. It is a conventional known small molecule with a molecular size of 914.19 Da.

2. Obtain the Protein to be Verified

FKBP12-V5-His sequence was obtained by linking 6×His and V5 tags to the sequence encoding FKBP12 protein respectively, in which 6×His tag was used for protein purification and V5 tag was used for Western blot detection. The FKBP12-V5-His sequence was constructed on pET28a vector and transformed into E. coli BL21 (DE3) strain. FKBP12 protein was obtained by adding IPTG at 18° C. overnight when OD600 was about 0.6 and purified by nickel column.

The biotinylated small molecule Rapamycin (2 μM), FKBP12 protein (5 μM) and SAₘ-Pup^{E} were mixed well and incubated at 30° C. for 20 min, then PafA7_{KR} (1 μM) and ATP (10 mM) were added into the system, mixed well and incubated at 30° C. for 6 h. Flag antibody was used for Western blot analysis. Compared with Rapamycin without biotinylation in the system, the FKBP12 protein bands in the experimental group migrated significantly. It shows that the system started working after connecting with the whole system through biotinylated Rapamycin, as shown in FIG. 21C.

There are many specific application ways of the invention, and the above is only a preferred embodiment of the invention. It should be noted that the above embodiments are only intended to illustrate the present invention and are not intended to limit the scope of protection of the present invention. To one of ordinary skill in the art, several modifications may be made without departing from the principles of the present invention, which should also be considered as the scope of protection of the present invention.

What is claimed is:

1. A system for detecting interactions between known molecules and proteins based on covalent linkages, comprising the following molecules:
a) a streptavidin-short peptide tetramer;
b) a PafA enzyme; and
c) biotin-modified known molecules.

2. The system for detecting the interactions between the known molecules and the proteins based on the covalent linkages according to claim 1, wherein a short peptide in the streptavidin-short peptide tetramer is a peptide chain containing 12-100 amino acids.

3. The system for detecting the interactions between the known molecules and the proteins based on the covalent linkages according to claim 2, wherein the short peptide comprises a Pup molecule or a mutant molecule of the Pup molecule, and a glutamine at an end of the Pup molecule is mutated into a glutamic acid, and wherein a sequence of which the Pup molecule is as shown in SEQ ID NO: 1; and
the mutation molecule of the Pup molecule is a Pup molecule with one or more mutations, and a sequence of the mutation molecule of the Pup molecule is shown by any sequence of SEQ ID NO: 2, SEQ ID NO: 3, and SEQ ID NO: 4.

4. The system for detecting the interactions between the known molecules and the proteins based on the covalent linkages according to claim 1, wherein characterized in that seven lysine mutations on a surface of the PafA enzyme are arginines, and mutation sites are K162R, K202R, K320R, K361R, K423R, K435R, and K446R.

5. The system for detecting the interactions between the known molecules and the proteins based on the covalent linkages according to claim 1, wherein the biotin-modified known molecules comprise any one or more of proteins, a DNA, an RNA, and small molecules.

6. The system for detecting the interactions between the known molecules and the proteins based on the covalent linkages according to claim 5, wherein the proteins comprise at least one of a protein, a peptide, a modified peptide, an antibody, and a lectin;
the RNA comprises at least one of a messenger RNA, a ribosome RNA, a long chain non-coding RNA, and a non-coding small RNA;
the DNA comprises at least one of a double-stranded DNA and a closed circular DNA; and
the small molecules comprise at least one of bioactive oligonucleotides, amino acids, vitamins, secondary metabolites of animal and plant microorganisms, and chemically synthesized small molecules in organisms.

7. A method for identifying interactions between known molecules and proteins by the system according to claim 1, comprising the steps of:
A. fully mixing the biotin-modified known molecules and a sample to be tested and incubating at 25° C.-35° C. for 0 h-1 h to obtain a first mixture;
B. adding a streptavidin-short peptide tetramer to the first mixture, thoroughly mixing, and incubating at 25° C. to 35° C. for 0 h-1 h to obtain a second mixture;
C. adding a PafA enzyme to the second mixture, thoroughly mixing, and incubating at 25° C.-35° C. for 1 min-6 h to obtain a third mixture;
D. adding a biotin-labeled affinity medium to the third mixture to isolate a streptavidin-short peptide and proteins connected by the streptavidin-short peptide; and
E. conducting a mass spectrometry identification.

8. The method for identifying the interactions between the known molecules and the proteins according to claim 7, wherein the sample to be tested comprises at least one of a protein, a living cell or tissue, a membrane protein, a cell lysate, and a tissue lysate.

9. A method for verifying an interaction between a known molecule and a protein by the system according to claim 1, comprising the steps of:
S1. mixing the known molecule to be verified with the protein to be verified, and incubating at 25° C. to 35° C. for 0 h-1 h to obtain a first mixture;
S2. adding a streptavidin-short peptide tetramer to the first mixture, thoroughly mixing, and incubating at 25° C. to 35° C. for 0 h-1 h to obtain a second mixture;
S3. adding a PafA enzyme to the second mixture, thoroughly mixing, and incubating at 25° C.-35° C. for 1 min-6 h; and
S4. conducting a western blot analysis to detect the interaction between the known molecule to be verified and the protein to be verified.

10. The method for verifying the interaction between the known molecule and the protein by the system according to claim 9, wherein the known molecule to be verified is a known molecule modified by a biotin and comprises any one or more of a protein, a DNA, a RNA, and small molecules;
the protein comprises at least one of a protein, a polypeptide, a modified peptide, an antibody, and a lectin;
the RNA comprises at least one of a messenger RNA, a ribosome RNA, a long chain non-coding RNA, and a non-coding small RNA;
the DNA comprises at least one of a double-stranded DNA and a closed circular DNA; and
the small molecules comprise at least one of bioactive oligonucleotides, amino acids, vitamins, secondary metabolites of animal and plant microorganisms, and chemically synthesized small molecules in organisms.


SEQUENCE LISTING (20 sequences)

SEQ ID NO: 1 - protein, 64 aa, Artificial Sequence
  The sequence is synthesized.
MAQEQTKRGGGGGDDDDIAGSTAAGQERREKLTEETDDLLDEIDDVLEENAEDFVRAYVQKGGE

SEQ ID NO: 2 - protein, 33 aa, Artificial Sequence
  The sequence is synthesized.
LTEETDDLLDEIDDVLEENAEDFVRAYVQRGGE

SEQ ID NO: 3 - protein, 28 aa, Artificial Sequence
  The sequence is synthesized.
DDLLDEIDDVLEENAEDFVRAYVQRGGE

SEQ ID NO: 4 - protein, 22 aa, Artificial Sequence
  The sequence is synthesized.
IDGLLENNGPIFAPSYVQYGGE

SEQ ID NO: 5 - protein, 178 aa, Artificial Sequence
  The sequence is synthesized.
MGAEAGITGTWYNQLGSTFIVTAGADGALTGTYESAVGNAESRYVLTGRYDSAPATDGSGTALGWTVAWRNNYRNAHSATTWSGQYVGGAEARINTQWLLTSGTTEANAWRSTLVGHDTFTRVRPSAASGGGGGSGSHHWGGGGSLTEETDDLLDEIDDVLEENAEDFVRAYVQRGGE

SEQ ID NO: 6 - protein, 461 aa, Artificial Sequence
  The sequence is synthesized.
MQRRIMGIETEFGVTCTFHGHRRLSPDEVARYLFRRVVSWGRSSNVFLRNGARLYLDVGSHPEYATAECDSLVQLVTHDRAGEWVLEDLLVDAEQRLADEGIGGDIYLFKNNTDSAGNSYGCHENYLIVRAGEFSRISDVLLPFLVTRQLICGAGKVLQTPKAATYCLSQRAEHIWEGVSSATTRSRPIINTRDEPHADAEKYRRLHVIVGDSNMSETTTMLKVGTAALVLEMIESGVAFRDFSLDNPIRAIREVSHDVTGRRPVRLAGGRQASALDIQREYYTRAVEHLQTREPNAQIEQVVDLWGRQLDAVESQDFAKVDTEIDWVIKRKLFQRYQDRYDMELSHPKIAQLDLAYHDIKRGRGIFDLLQRKGLAARVTTDEEIAEAVDQPPQTTRARLRGEFISAAQEAGRDFTVDWVHLKLNDQAQRTVLCKDPFRAVDERVKRLIASMGGGHHHHHH

SEQ ID NO: 7 - protein, 461 aa, Artificial Sequence
  The sequence is synthesized.
MQRRIMGIETEFGVTCTFHGHRRLSPDEVARYLFRRVVSWGRSSNVFLRNGARLYLDVGSHPEYATAECDSLVQLVTHDRAGEWVLEDLLVDAEQRLADEGIGGDIYLFKNNTDSAGNSYGCHENYLIVRAGEFSRISDVLLPFLVTRQLICGAGKVLQTPRAATYCLSQRAEHIWEGVSSATTRSRPIINTRDEPHADAERYRRLHVIVGDSNMSETTTMLKVGTAALVLEMIESGVAFRDFSLDNPIRAIREVSHDVTGRRPVRLAGGRQASALDIQREYYTRAVEHLQTREPNAQIEQVVDLWGRQLDAVESQDFARVDTEIDWVIKRKLFQRYQDRYDMELSHPKIAQLDLAYHDIRRGRGIFDLLQRKGLAARVTTDEEIAEAVDQPPQTTRARLRGEFISAAQEAGRDFTVDWVHLRLNDQAQRTVLCRDPFRAVDERVRRLIASMGGGHHHHHH

SEQ ID NO: 8 - RNA, 18 nt, Artificial Sequence
  The sequence is synthesized.
cgucucggcucggcugcu

SEQ ID NO: 9 - DNA, 22 bp, Artificial Sequence
  The sequence is synthesized.
cggcagatgcataacaaaggtg

SEQ ID NO: 10 - DNA, 22 bp, Artificial Sequence
  The sequence is synthesized.
cacctttgttatgcatctgccg

SEQ ID NO: 11 - DNA, 16 bp, Artificial Sequence
  The sequence is synthesize.
cctttgttatgcaaat

SEQ ID NO: 12 - DNA, 11 bp, Artificial Sequence
  The sequence is synthesized.
atatgcaaatt

SEQ ID NO: 13 - DNA, 85 bp, Artificial Sequence
  The sequence is synthesized.
catggatccacgctatcaacgtaatgtcgaggccgtcaacgagatgtcgacactatcgacacgtagtaagctgccagatgacaaa

SEQ ID NO: 14 - DNA, 85 bp, Artificial Sequence
  The sequence is synthesized.
catggatccacgctatcaacgtaatgtcgaggccgtcaacaagataagccccctatcgacacgtagtaagctgccagatgacaaa

SEQ ID NO: 15 - DNA, 25 bp, Artificial Sequence
  The sequence is synthesized.
ttgaccacatggaccaaacagtctg

SEQ ID NO: 16 - DNA, 25 bp, Artificial Sequence
  The sequence is synthesized.
ttgaccacatagaccgactggtcta

SEQ ID NO: 17 - DNA, 17 bp, Artificial Sequence
  The sequence is synthesized.
caacccatgagtcatac

SEQ ID NO: 18 - protein, 23 aa, Artificial Sequence
  The sequence is synthesized.
GGSGDRRRFNSADYKGPRRRKAD

SEQ ID NO: 19 - protein, 22 aa, Artificial Sequence
  The sequence is synthesized.
GGSGDRRFNSADYKGPRRRKAD

SEQ ID NO: 20 - protein, 23 aa, Artificial Sequence
  The sequence is synthesized.
GGSGDRRRFNSADYKAPRRRKAD